(19) United States
(12) Patent Application Publication
(10) Pub. No.: US 2013/0283463 A1
(43) Pub. Date: Oct. 24, 2013

(54) CUCURBITA PEPO PUMPKINS HAVING A MUTANT ALLELE FOR POWDERY MILDEW RESISTANCE

(71) Applicant: Hollar Seeds, Rocky Ford, CO (US)

(72) Inventor: QI ZHANG, Rocky Ford, CO (US)

(21) Appl. No.: 13/933,289

(22) Filed: Jul. 2, 2013

Related U.S. Application Data
(63) Continuation-in-part of application No. 12/878,220, filed on Sep. 9, 2010.

(51) Int. Cl. A01H 5/08

(52) U.S. Cl. CPC A01H 5/08
USPC 800/265; 800/307; 435/410

(57) ABSTRACT

The present invention relates to a novel mutant allele of Cucurbita pepo L. (pumpkin) designated Pm2, which confers a strong and consistent powdery mildew resistance to Cucurbita pepo plants. The present invention also relates to plants and seeds of the family or line carrying the Pm2 allele. In addition, the present invention is also directed to transferring the Pm2 allele to plants in the same species lacking the allele, and is useful for producing novel types and varieties of powdery mildew resistant Cucurbita pepo.

### CROSS-REFERENCE TO RELATED APPLICATIONS

This application is a Continuation-In-Part of and claims the benefit of priority under 35 U.S.C. §119 from U.S. application Ser. No. 12/878,220, filed Sep. 9, 2010, which is incorporated herein in its entirety by reference.

### BACKGROUND OF THE INVENTION

The present invention relates to Cucurbita pepo L. (pumpkin) plants and plant parts having a mutant allele designated Pm2, which confers a strong and consistent powdery mildew resistance to Cucurbita pepo plants. The present invention also relates to plants and seeds of the family or line carrying the Pm2 allele. In addition, the present invention is also directed to transferring the Pm2 allele to plants in the same species lacking the allele, and is useful for producing novel plants and varieties of powdery mildew resistant Cucurbita pepo. All publications cited in this application are herein incorporated by reference.

The genus Cucurbita is composed of five species: C. pepo, C. maxima, C. moschta, C. mixta and C. ficifolia. The terms “pumpkin”, “squash” and “gourd” cannot be directly related to the species; forms of several are called pumpkins and the same is true of squashes and gourds. The term “pumpkin” is normally applied to the edible fruit of any species of Cucurbita utilized when ripe as a table vegetable, in pies, or as an ornamental. The term “squash” was evidently derived from a north-eastern American Indian word indicating a fruit, apparently Cucurbita pepo L., eaten raw as an immature fruit or consumed for the mature seed (T. W. Whitaker 1986. Breeding Vegetable Crops, pp. 210-223). It is now also applied to certain baking cultivars of C. pepo (e.g., Acorn), C. moschta (e.g., butternut), C. mixma (e.g., Orange Banana) and C. mixta (e.g., Cushaw) that are used in the mature state. The terms “pumpkin”, “squash” and “gourd” are confined to the species C. pepo. Pepo is an extremely large and diverse species in the genus Cucurbita which is separated into four categories: Halloween pumpkin, summer squash, winter squash and ornamental gourd. Each category is composed of many cultural types and has many varieties over the world. The pepo crops are widely cultivated and play a significant role in human nutrition and economic development globally.

A common disease of C. pepo crops is powdery mildew, which is one of the main limiting factors of pumpkin, squash and gourd production around the world. It would be commercially very desirable to have new varieties of C. pepo that have a better and more consistent resistance to powdery mildew for growers, the commercial market, and especially for organic growers.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

According to the present invention, there is provided a novel pumpkin mutant allele designated Pm2 that confers increased and consistent powdery mildew resistance to Cucurbita pepo plants. This invention thus relates to C. pepo seeds having mutant allele Pm2, C. pepo plants having mutant allele Pm2, C. pepo varieties and C. pepo hybrids having mutant allele Pm2, and to a method for producing a C. pepo plant having mutant allele Pm2.

It is a further aspect of the present invention to provide a method of producing a pumpkin plant having increased powdery mildew resistance by crossing a pumpkin plant having mutant allele Pm2 with itself or another pumpkin variety, any such methods may include selfing, backcrosses, hybrid production, crosses to populations, and the like. Thus, all pumpkin plants and plant parts having increased powdery mildew resistance that are derived from a pumpkin plant having mutant allele Pm2 are within the scope of this invention.

This invention also relates to methods for the creation of variants by mutagenesis or transformation of pumpkin plants having mutant allele Pm2.

In another aspect, the present invention provides regenerable cells for use in tissue culture of C. pepo pumpkin plants having mutant allele Pm2. The tissue culture will preferably be capable of regenerating plants having the physiological and morphological characteristics of the foregoing C. pepo plant, and of regenerating plants having substantially the same genotype as the foregoing C. pepo plant. Preferably, the regenerable cells in such tissue cultures will be embryos, protoplasts, meristematic cells, callus, pollen, leaves, anthers, pistils, stems, petioles, roots, root tips, fruits, seeds, flowers, cotyledons, hypocotyls or the like. Still further, the present invention provides C. pepo plants regenerated from the tissue cultures of the invention.

The invention further provides for a C. pepo plant having mutant allele Pm2 having consistent increased resistance over the growing season to powdery mildew and exhibiting a mean colony count of powdery mildew, wherein said increased resistance is exhibiting at least about 75%, 73%, 72%, 65%, 60%, 59%, 58%, 55%, 53%, 50%, 47%, 45%, 42%, 38%, 36%, 33%, 31%, 29%, 28%, 27% and 25% fewer mean powdery mildew colony counts per plant when compared to commercial pumpkin varieties in an environment having a level of powdery mildew present.

In addition to the exemplary aspects and embodiments described above, further aspects and embodiments will become apparent by study of the following descriptions.

### BRIEF DESCRIPTION OF THE DRAWINGS

Exemplary embodiments are illustrated in the drawings. It is intended that the embodiments and figures disclosed herein are to be considered illustrative rather than limiting. For a detailed description of various embodiments, reference will now be made to the accompanying illustrative drawings in which:

FIG. 1 shows agarose gel electrophoresis of alleles for PCR primer set AgBPMR5F1/R1. From left to right: DNA molecular weight 100 bp marker ladder, followed by two lanes each for M Line 411, HSPMR7B1, and Hdy 3111, respectively.

FIG. 2 shows a DNA sequence alignment of the PCR products from primer set AgBPMR5F1/R1 for M Line 411, which is derived from commercial variety Merlin and contains the existing PMR allele Pm, HSPMR7B1, which contains mutant allele Pm2 of the present invention, and Hdy 3111, a powdery mildew susceptible line.

FIG. 3 shows the sequences produced from the reverse primer of AgBPMR5F1/R1 for M Line 411, which is derived from commercial variety Merlin and contains the existing PMR allele Pm (SEQ ID NO:1), HSPMR7B1, which contains mutant allele Pm2 of the present invention (SEQ ID NO:2), and Hdy 3111, a powdery mildew susceptible line (SEQ ID NO:3).

### SUMMARY OF THE SEQUENCE LISTING

The present application is being filed along with a Sequence Listing in electronic format. The Sequence Listing is entitled SequenceListing_ST25.txt, was created on 20 Jun. 2013 and is 3 kb in size. The information in the electronic format of the Sequence Listing is part of the present application and is incorporated herein by reference in its entirety.

SEQ ID NO:1 sets forth the sequence from the reverse primer of AgBPMR5F1/R1 for M Line 411 (Pm).

SEQ ID NO:2 sets forth the sequence from the reverse primer of AgBPMR5F1/R1 for HSPMR7B1 (Pm2).

SEQ ID NO:3 sets forth the sequence from the reverse primer of AgBPMR5F1/R1 for Hdy 3111 (pm).

### DETAILED DESCRIPTION OF THE INVENTION

In the description and tables that follow, a number of terms are used. In order to provide a clear and consistent understanding of the specification and claims, including the scope to be given such terms, the following definitions are provided:

Allele. An “allele” is any of one or more alternative form of a gene, all of which relate to one trait or characteristic. In a diploid cell or organism, the two alleles of a given gene occupy corresponding loci on a pair of homologous chromosomes.

Amplicon. A piece of DNA formed as the product of natural or artificial amplification events.

Backcross. “Backcross” is a breeding method in which a breeder repeatedly crosses hybrid progeny back to one of the parents, for example, a first generation hybrid F₁ with one of the parents of the F₁ hybrid.

Commercial pumpkin. “Commercial pumpkin” means a pumpkin (C. pepo) that has been commercially sold. A commercial pumpkin is a pumpkin plant that does not contain the PMR allele Pm2.

Complete growing season. As used herein, “complete growing season” means from the C. pepo planting date until the date that 95% of C. pepo leaves have died naturally and have not died due to disease.

Consistent resistance. As used herein, “consistent resistance” refers to C. pepo plants expressing resistance to powdery mildew for the complete growing season when powdery mildew is present in the environment.

Cucurbita pepo. An extremely large and diverse species in the genus Cucurbita which is separated into four categories: Halloween pumpkin, summer squash, winter squash and ornamental gourd. Each category is composed of many cultural types and has many varieties over the world. The C. pepo plants containing mutant allele Pm2 of the present invention are pumpkin plants.

Dominant inheritance. Refers to a mode of inheritance in which the phenotype of a certain characteristic or trait is determined by a dominant allele.

Dominant mutation. The phenotype of a dominant mutation is visible in a heterozygous genotype.

Essentially all the physiological and morphological characteristics. A plant having “essentially all the physiological and morphological characteristics” means a plant having the physiological and morphological characteristics, except for the characteristics derived from the converted gene.

Gene converted (conversion). “Gene converted” or “Single gene converted” (or conversion) plant refers to plants which are developed by a plant breeding technique called backcrossing wherein essentially all of the desired morphological and physiological characteristics of an inbred are recovered in addition to the one or more genes transferred into the inbred via the backcrossing technique or via genetic engineering.

Genetic transformation. Refers to the genetic alteration of a cell resulting from the uptake, genomic incorporation, and expression of foreign genetic material.

ICUGI. International Cucurbit Genomic Initiative, hosting a Cucurbit genomics database website containing information on functional genomics, mapping and bioinformatics for different melon genotypes and tissues. www.icugi.org.

Increased resistance. “Increased resistance” means improved resistance to powdery mildew caused by Sphaerotheca fuliginea Poll. and/or Erysiphe cichoracearum DC when compared to/versus commercial pumpkin varieties. That is, the plant's resistance can be highly resistant to powdery mildew, which means the plants have few or no colony counts of powdery mildew on any plant parts. Additionally, increased resistance means that when compared to/versus commercial pumpkin varieties, the varieties of the present invention which contain the PMR mutant allele have lower colony counts of powdery mildew on their plant parts than the plant parts of the commercial pumpkin varieties. The increased resistance reflects the capability of a plant to reduce the establishment of an infection and may, for example, be calculated by determining the success of the plant surviving upon contact with the infectious agent (i.e., the powdery mildew fungus). Increased resistance is also intended to mean that the disease symptoms caused by powdery mildew are minimized or lessened.

Indels. A term for the insertion or deletion of bases in the DNA of an organism.

Leaf. “Leaf” means an above-ground plant organ specialized for photosynthesis and transpiration. For these purposes, a leaf is typically flat (laminar) and thin, to expose the cells containing chloroplast to light over a broad area, and to allow light to penetrate fully into the tissues and to allow heat to diffuse optimally.

Mean colonies per square inch. As used herein, “mean colonies per square inch” means the mean count of the number of powdery mildew colonies on a square inch of plant parts, including the leaf, petiole and stems. The fungal colonies are formed by conidia.

Mutation. Mutations are changes in the DNA sequence of a cell's genome and are caused by mutagens, like radiation or chemicals, as well as by errors that occur during DNA replication.

Pedigree breeding/selection. “Pedigree breeding” is a breeding method used during the inbreeding of populations of self- and cross-pollinated species for the development of desirable homogeneous lines. Pedigree selection generally begins with an F₂ population and continues until homogeneous lines are developed.

Petiole. “Petiole” means the stalk of a leaf, attaching the leaf blade to the stem.

Plant. “Plant” includes plant cells, plant protoplasts, plant cells of tissue culture from which C. pepo plants can be regenerated, plant calli, plant clumps and plant cells that are intact in plants, or parts of plants such as pollen, flowers, seeds, leaves, stems, rind, flesh and the like.

Pm2. “Pm2” refers to the mutant allele of the powdery mildew resistant gene of the present invention which confers an increased and more consistent powdery mildew resistance to C. pepo plants than the existing allele Pm in the common commercial C. pepo varieties, wherein a sample of seed containing Pm2 was deposited under ATCC Accession Number PTA-8167.

PMR (Powdery Mildew Resistant). “PMR” refers to a C. pepo plant phenotypically displaying a level of powdery mildew resistance. The level of powdery mildew on plants is divided into six PMR scores or categories consisting of 0, 1, 2, 3, 4, 5, in which 0 indicates that the plants are completely resistant to the disease and there are no symptoms of the disease on the plants at all, and a rating of 5 indicates that the plants are 100% infected and susceptible.

PMR5. Refers to the gene powdery mildew resistance 5. In Arabidopsis, identified as locus AT5G58600 with a role in resistance to powdery mildew infection. The ortholog in Cucurbita pepo is identified as unigene PU055704.

PMS (Powdery Mildew Susceptible). “PMS” refers to a C. pepo plant phenotypically displaying powdery mildew susceptibility.

Quantitative Trait Loci (QTL). “Quantitative trait loci (QTL)” refer to genetic loci that control to some degree numerically representable traits that are usually continuously distributed.

Regeneration. “Regeneration” refers to the development of a plant from tissue culture.

SNP. Refers to single nucleotide polymorphisms. Genetic variation in a DNA sequence that occurs when a single nucleotide in a genome is altered; SNPs are usually considered to be point mutations that have been evolutionarily successful enough to recur in a significant proportion of the population of a species.

Stem. “Stem” means the above ground structures that have vascular tissue and that support, for example, leaves, flowers, seed, fruit, etc. The stem is normally divided into nodes and internodes, the nodes hold buds which grow into for example, one or more leaves, inflorescence (flowers), cones or other stems (or branches), while the internodes act as spaces that distance one node from another.

Transcriptome. The full range of messenger RNA, or mRNA, molecules expressed by an organism.

Unigene. An individual unigene set is defined as a group of transcript sequences that, based on sequence homology, originate from the same gene or expressed pseudogene.

Yield. “Yield” means the total weight in kilograms of marketable harvested fruit from an experimental plot or field.

Cucurbita pepo mutant allele Pm2 of the present invention confers increased and consistent powdery mildew resistance to pumpkin plants. As used herein powdery mildew resistance refers to pumpkin plants that display little to no powdery mildew symptoms throughout the growing season.

Mutant allele Pm2 of the present invention is heritable and has been transferred to several different pumpkin lines. Pumpkins having mutant allele Pm2 have shown uniformity and stability.

A common disease of C. pepo crops is powdery mildew which is largely caused by two different fungi: Sphaerotheca fuliginea Poll. and Erysiphe cichoracearum DC. Powdery mildew epidemics are ubiquitous and often result in considerable losses to fields of pumpkin, squash and gourd. Powdery mildew infections on the crops appear as a white powdery growth that develops on the leaf blades, peduncles, and stems of susceptible plants. Usually infection begins on older leaves and then spreads to other parts of the plant. It frequently compromises yield by reducing the leaf area available for photosynthesis, accelerating senescence, and, ultimately, killing the infected plants. Therefore, the disease is one of the main limiting factors of pumpkin, squash and gourd production around the world.

To solve the powdery mildew disease problem, breeding for powdery mildew resistance has been conducted world-wide for a number of years. There is an existing powdery mildew resistant gene (Pm) in Cucurbita pepo that was derived from an interspecific breeding process which was conducted by Cornell University researchers, including Dr. Maximo E. Contin in the time period of 1970s-1990s. After the gene was transferred to C. pepo from two wild Cucurbita species, C. ecuadorensis and C. martinezii, some C. pepo lines with PMR were developed by the end of 1990s. The moment the PMR lines were released, all of the seed companies in America started to integrate the Pm allele into their C. pepo materials; which include the C. pepo summer squash, winter squash and pumpkins. All powdery mildew resistant C. pepo varieties developed recently contain this allele, which includes the pumpkin varieties discussed in our patent application such as Merlin, Magic Lantern and Spartan. The alleles compared with Pm2 in the application are Pm and a recessive allele pm. The most common commercially available pumpkin and squash varieties have powdery mildew resistance which is believed to be conferred by a single co-dominant gene (See M. T. Mcgrath. 2004. Managing powdery mildew in winter squash with genetic control and chemical control. Plant Pathology, Cornell Univ. Pub. No. P-2004-0016-Nea; and R. Cohen et. al. 2004. Single-gene resistance to powdery mildew in zucchini squash (Cucurbita pepo L.) Euphytica, Vol. 130 (3):433-441). The heterozygous plants carrying the co-dominant gene produce a segregating ratio of 1 powdery mildew resistant plant to 2 powdery mildew tolerant plants to 1 powdery mildew susceptible plant. C. pepo varieties currently available exhibit a range in the level of resistance depending on whether the variety is heterozygous or homozygous for the co-dominant gene. None of the currently available commercial pumpkin varieties maintain a consistent powdery mildew resistance level during the complete growing season, in contrast to the Pm2 mutant allele of the present invention which provides consistent powdery mildew resistance over a complete growing season. Varieties homozygous for currently available co-dominant gene usually develop somewhat less powdery mildew than those heterozygous for currently available co-dominant gene. In catalogues these varieties are often described as resistant and tolerant, respectively.

All currently available pumpkin and squash varieties designated as being powdery mildew resistant or tolerant exhibit some suppression of powdery mildew development for a few weeks, but become affected by different levels of disease severity and may die up to a month or more before the end of the complete growing season. Effective powdery mildew chemical control is therefore still needed in order to get fruit and seed production. Although chemicals such as Tosin M, Nova, FLINT, AMISTAR, QUADRIS, QUINTEC, DMI, BAYLETON, PROCURE, etc., have been used to effectively manage powdery mildew for decades, unfortunately, all such materials are at risk for the development of resistance by the various powdery mildew strains. In view of the inevitable occurrence of powdery mildew and the significant expense for disease control, breeding for better powdery mildew resistance in pumpkin and squash is very important.

Unexpectedly, the novel genetic factor of the present invention, which has been designated Pm2, is capable of transmitting increased and consistent powdery mildew resistance and has been determined to be a novel single dominant allele that may have additional modifier genes. It is a feature of the present invention that mutant allele Pm2 may be used in and transferred among the various pumpkin, squash and gourd varieties in the C. pepo species.

The present invention is directed to developing unique plants of Cucurbita pepo containing mutant allele Pm2 that have strong and consistent resistance to powdery mildew. The pumpkin fruit and plants of the present invention unexpectedly express an important and substantial increase in powdery mildew resistance. The transferable mutant allele Pm2 that conveys this characteristic has been isolated and incorporated into various genetic backgrounds, including HSPMR7B1, HSR4706, HSR4707, HSR4709, HSR4710, HSR4711, HSR4720, HSR4721 and HSR4722. The mutant allele of the instant invention, Pm2, has also been expressed in different genetic backgrounds of pumpkin. To date, no commercialized C. pepo variety has consistent resistance to powdery mildew, in contrast to pumpkin plants containing mutant allele Pm2 of the present invention. Crosses with the mutant allele Pm2 of the present invention unexpectedly expressed a unique powdery mildew resistance pattern over multiple years of trials, in which the plants showed a consistent strong resistance level throughout the whole growing season and without the aid of any chemical application.

As used herein, the term plant includes plant cells, plant protoplasts, plant cell tissue cultures from which C. pepo plants can be regenerated, plant calli, plant clumps and plant cells that are intact in plants, or parts of plants, such as embryos, pollen, ovules, flowers, stems, leaves, roots, root tips, anthers, pistils, and the like.

### EXAMPLES

The following examples are provided to further illustrate the present invention and are not intended to limit the invention beyond the limitations set forth in the appended claims.

### Example 1

### Development of the PMR Mutant Allele Pm2 in C. pepo Plants

The mutant allele of the present invention, Pm2, unexpectedly arose from an open pollinated pumpkin plant from a research field in 2000. The series of crosses and selections is shown in Table 1. [(Howden×Coach)×Sprit] in Table 1 was a pumpkin three-way cross made between 1999 and 2000. None of these three parents, Hamden, Coach or Sprit, have the resistance of the mutant allele Pm2 of the present invention. The H7B-1Lop population, considered an F₂, was derived from a plant which exhibited strong powdery mildew resistance that was open-pollinated in the three-cross generation in the summer of 2000. During a pedigree selection process, five plants which exhibited a high level of powdery mildew resistance were selected and selfed from the F₂ population in 2001 and used to form the F₃ families H7B-1LOP-1, H7B-1LOP-2, H7B-1LOP-3, H7B-1LOP-4, and H7B-1LOP-5, respectively. In the summer of 2002, it was found that families H7B-1LOP-2, H7B-1LOP-3 and H7B-1LOP-5 all produced ratios of 3 powdery mildew resistant plants to 1 powdery mildew susceptible plant, while all the plants in the H7B-1LOP-1 family were powdery mildew resistant. Four powdery mildew plants in the H7B-1Lop-1 family and one in the H7B-1Lop-4 family were further selfed and used to form the H7B-1Lop-1-1, H7B-1Lop-1-2, H7B-1Lop-1-3, H7B-1Lop-1-4 and H7B-1Lop-4-1 families. The F₄ seeds were planted and the seedlings were screened with powdery mildew innocula (Sphaerotheca fuliginea Poll. and Erysiphe cichoracearum DC). The powdery mildew screening showed that all five families were non-segregating for the powdery mildew resistance trait in the fall of 2002. The F₅ lines, H7B-1Lop-1-1-1, H7B-1Lop-1-1-2, H7B-1Lop-1-1-3 and H7B-1Lop-4-1-1 were produced from the F₄ families H7B-1Lop-1-1 and H7B-1Lop-4-1 in the same season. At the same time two crosses were also made by crossing F₄ (H7B-1Lop-1-1) plants to a common powdery mildew susceptible pumpkin male designated 710M. Additionally, two backcrosses (BC) were made to produce BC populations for further testing during the winter of 2002. The backcrosses were (H7B-1Lop-1-1×710M)BC11 and (H7B-1Lop-1-1×710M)BC12.

Table 1 shows a summary of the series of crosses described in the previous paragraph. Column 1 shows the pedigree, column 2 shows the year for each cross, and column 3 shows remarks concerning each cross.

TABLE 1

| Pedigree | Year | Remarks |
|---|---|---|
| Recombining Generation | | |
| [(Howden × Coach) × Sprit]-1L | 1999- | Designated H7B-1L |
| | 2000 | |
| O.P. or F₂ Population | | |
| H7B-1Lop | 2001 | O.P. plant with strong PMR |
| F₃ Family | | |
| H7B-1Lop-1 | 2002 | No PMR Segregation |
| H7B-1Lop-2 | 2002 | PMR segregation, 3PMR:1PMS |
| H7B-1Lop-3 | 2002 | PMR segregation, 3PMR:1PMS |
| H7B-1Lop-4 | 2002 | No PMR segregation |
| H7B-1Lop-5 | 2002 | PMR segregation, 3PMR:1PMS |
| F₄ Family | | |
| H7B-1Lop-1-1 | 2002 | No PMR segregation |
| H7B-1Lop-1-2 | 2002 | No PMR segregation |
| H7B-1Lop-1-3 | 2002 | No PMR segregation |
| H7B-1Lop-1-4 | 2002 | No PMR segregation |
| H7B-1Lop-4-1 | 2002 | No PMR segregation |
| F₅ Family | | |
| H7B-1Lop-1-1-1 | 2003 | No PMR segregation |
| H7B-1Lop-1-1-2 | 2003 | No PMR segregation |
| H7B-1Lop-1-1-3 | 2003 | No PMR segregation |
| H7B-1Lop-4-1-1 | 2003 | No PMR segregation |
| Test Crosses & Backcrosses | | |
| H7B-1Lop-1-1 × 710M | 2002 | |
| (H7B-1Lop-1-1 × 710M)BC11 | 2002 | PMR segregation, 1PMR:1PMS |
| (H7B-1Lop-1-1 × 710M)BC12 | 2002 | PMR segregation, 1PMR:1PMS |
| F₁ Hybrids | | |
| H7B-1Lop-1-1-1 × BSP1 | 2002 | Designated HSR4706, PMR |
| H7B-1Lop-1-1-1 × GDF41 | 2002 | Designated HSR4707, PMR |
| H7B-1Lop-1-1-1 × HDD31 | 2002 | Designated HSR4709, PMR |

### Example 2

### Determining the Genetic Relation of the PMR Mutant Allele Pm2 in the Invention and Powdery Mildew Susceptible Allele Pm in Commercial Varieties

To determine the genetic mechanism of the new powdery mildew resistance characteristic of the present invention, five F₃ families were produced and planted in the summer of 2002. These five F₃ families were produced by selecting and selfing five plants from the F₂ population in 2001. The F₃ families produced from the selected five plants were designated H7B-1LOP-1, H7B-1LOP-2, H7B-1LOP-3, H7B-1LOP-4, and H7B-1LOP-5, respectively. It was found that families H7B-1LOP-2, H7B-1LOP-3 and H7B-1LOP-5 all produced ratios of 3 powdery mildew resistant plants to 1 powdery mildew susceptible plant however plants in the H7B-1LOP-1 family were all powdery mildew resistant. Upon comparison of comprehensive characteristics among the families, it was found that families H7B-1Lop-2, H7B-1Lop-3 and H7B-1Lop-5 were not ideal in either fruit traits or plant habits and thus, these families were not used in further breeding efforts. The H7B-1LOP-1 family or line was named as HSPMR7B1.

The two remaining families, H7B-1Lop-1 and H7B-1Lop-4, had desirable fruit characteristics, therefore four plants of the H7B-1Lop-1 family and one plant of the H7B-1Lop-4 family were selected and selfed to form H7B-1Lop-1-1, H7B-1Lop-1-2, H7B-1Lop-1-3, H7B-1Lop-1-4 and H7B-1Lop-4-1, respectively. The resulting F₄ seeds were planted and the seedlings were screened with powdery mildew inocula (Sphaerotheca fuliginea Poll. and Erysiphe cichoracearum DC). The powdery mildew screening showed that all five F₄ families were non-segregating for the powdery mildew resistance trait in the fall of 2002. Of the F₄ families, two were kept for advanced selections of other traits except for powdery mildew. The F₅ families, H7B-1Lop-1-1-1, H7B-1Lop-1-1-2, H7B-1Lop-1-1-3 and H7B-1Lop-4-1-1 were produced from the 2002 season. At the same time two crosses were also made by crossing F₄ (H7B-1Lop-1-1) plants to a common powdery mildew susceptible pumpkin male designated 710M. Additionally, two backcrosses (BC) were made to produce BC populations for further testing during the winter of 2002. The backcrosses were (H7B-1Lop-1-1×710M)BC11 and (H7B-1Lop-1-1×710M)BC12. The two BC populations were tested and both segregated 1 powdery mildew resistant plant to 1 powdery mildew susceptible plant during the summer 2003.

The data, as shown in Table 2, indicated that, except for the H7B-1LOP-1 family, all four other families had a heterozygous genotype and that the powdery mildew resistance phenotype of the present invention is under the control of a single dominant allele designated Pm2, and which may have some additional modifier genes in different genetic backgrounds. Table 2 shows the genetic mechanism of the new powdery mildew characteristic. Column 1 shows the pumpkin family, column 2 shows the number of plants in that family displaying the powdery mildew resistant phenotype, column 3 shows the number of plants in that family displaying the powdery mildew susceptible phenotype, and columns 4 and 5 show the Chi-square analysis of the data. “z” means that all X² are non significant at 0.01 probability and the PMR and PMS segregation meet 3:1 and “-- --” means not tested.

TABLE 2
PMR Segregation In Six Selfed And BC Families

| | | | Chi-square^{z} | |
|---|---|---|---|---|
| Family | PMR | PMS | χ²(3:1) | χ²(1:1) |
| F₃ Families | | | | |
| H7B-1Lop-1 | 55 | 0 | -- -- | -- -- |
| H7B-1Lop-2 | 37 | 13 | 0.0267 | -- -- |
| H7B-1Lop-3 | 47 | 16 | 0.0053 | -- -- |
| H7B-1Lop-4 | 28 | 9 | 0.0090 | -- -- |
| H7B-1Lop-5 | 31 | 11 | 0.032 | -- -- |
| BC Populations | | | | |
| (H7B-1Lop-1-1 × 710M)BC11 | 19 | 17 | -- -- | 0.1111 |
| ((H7B-1Lop-1-1 × 710M)BC12 | 24 | 27 | -- -- | 0.1765 |

### Example 3

### Determining the Genetic Relation of the PMR Mutant Allele Pm2 in the Present Invention and an Existing Powdery Mildew Resistant Allele Pm in Commercial Varieties

The genetic relation between Pm and Pm2 was also tested in 2008 and 2009. Three crosses were made in the greenhouse in the spring of 2008 with a descendant line of the present invention HSPMR7B1, “H7BFlat-1”, carrying Pm2, and three lines lodged with Pm, MlnF411, MltnF42-1 and HKN89-3R-1(op)-1-1, which were derived from the commercial varieties Merlin, Magic Lantern and a breeding material of Cornell University. The three crosses, H7BFlat-1×MlnF411, H7BFlat-1×MltnF42-1 and H7BFlat-1×HKN89-3R-1(op)-1-1, were tested in Hollar's 2008 summer trial in Rocky Ford, Colo. The experiment was completely randomized with two replications, in which the aforementioned four parental lines and a commercial powdery mildew susceptible variety, Frosty, were taken as comparisons. All the experimental entries were planted on May 15 and observation on powdery mildew was not made until August because the disease would usually not occur in the fields during the early growing season. The incidence of powdery mildew on plants was divided into six scores or categories consisting of 0, 1, 2, 3, 4, 5, in which 0 indicates that the plants are completely resistant to the disease and there are no symptoms of the disease on the plants at all; and a rating of 5 indicates that the plants are 100% infected and susceptible. The powdery mildew resistance data was collected on Sep. 10, 2008 when the powdery mildew symptom of the pumpkin plants was most distinguishable. Compared to the powdery mildew resistance among all entries, H7BFlat-1, H7BFlat-1×MlnF411 and H7BFlat-1×MltnF42-1 had the strongest resistance that almost reached an immune level; the parental lines MlnF411, MltnF42-1 and HKN89-3R-1(op)-1-1 showed a significantly lower level than the former three, but are still in a strong resistant category; the resistance of the cross H7BFlat-1×HKN89-3R-1(op)-1-1 was on the strong side of the intermediate level of its parent lines; and Frosty had completely come down with the powdery mildew disease (Table 3). At the same time, the F₂ seeds of the three crosses were generated in the Hollar green house. The resistant segregation of the three populations was further tested in the greenhouse in the fall of 2008 and the summer of 2009 (Table 4). Although the difference of the powdery mildew resistant levels between the plants of the present invention containing the Pm2 mutant allele (Pm2Pm2 and Pm2Pm) and the common resistant plants (PmPm) was much smaller than in between all the resistant plants (Pm2Pm2, Pm2Pm and PmPm) and the susceptible comparison (pmpm), the test result showed that the inheritance of Pm2, Pm and pm is similar to the genetic mechanism of C. pepo fruit color genes in which a dominant white color gene (W) is dominant to a green color (C) which is further dominant to a recessive white color (w) and etc. In spite of variations of the F₂ populations in the greenhouse and field, the Chi-square scores indicated that the segregation ratio of the PMR mutant plants (Pm2Pm2 and Pm2Pm) and the common resistant plants (PmPm) in all tested F₂ population is not significant from 3:1 at 0.01 level. One conclusion is that the mutant allele Pm2 is dominant to the Pm, and that Pm2 and Pm are both dominant to the recessive allele pm. In Table 3, “y” means the incidence of powdery mildew is divided five levels in which 0 stands for complete resistance (or immunity) and 5 for complete susceptibility and “z” means all means with the same letter are not significantly different; those with the different letters are significantly different at 0.01 probability. In Table 4, “z” means that all X² are non significant at 0.01 probability and the Pm2Pm2 and PmPm segregation pattern is typical of a single dominant allele.

TABLE 3
Assessment Of Pm2 And Pm Expression In The Parent Lines And F₁ In 2008

| | | Incidence of powdery mildew^{y} | |
|---|---|---|---|
| Tested Entries | Replication | (20 plants) | Mean^{Z} |
| Frosty | I | 5.0 4.5 5.0 5.0 5.0 5.0 4.5 5.0 5.0 5.0 | 4.9375a |
| (pmpm) | | 4.5 5.0 5.0 5.0 5.0 5.0 5.0 5.0 5.0 5.0 | |
| | II | 5.0 5.0 5.0 5.0 5.0 5.0 5.0 5.0 5.0 5.0 | |
| | | 5.0 5.0 5.0 4.5 5.0 5.0 5.0 5.0 4.5 5.0 | |
| MltnF42-1 | I | 2.0 2.5 2.5 2.5 3.0 3.0 1.5 2.5 2.5 2.0 | 2.3625b |
| (Pm line) | | 1.5 1.5 3.0 2.5 2.0 2.0 2.5 2.0 2.0 2.5 | |
| | II | 2.5 3.0 2.0 3.0 2.5 2.0 2.5 3.0 2.5 2.0 | |
| | | 3.0 2.5 2.5 2.0 2.0 2.5 2.5 2.0 2.0 3.0 | |
| MlnF411 | I | 2.5 1.5 2.5 1.5 2.0 2.5 1.5 2.0 2.0 2.0 | 2.3250b |
| (Pm line) | | 1.5 3.0 1.5 2.0 2.5 2.0 2.0 2.5 1.0 2.0 | |
| | II | 2.0 2.5 2.5 1.5 2.0 1.5 2.5 2.5 3.0 1.5 | |
| | | 1.5 2.0 2.5 2.5 2.5 2.0 1.5 2.0 2.5 2.5 | |
| HKN89-3R-1(op)-1-1 | I | 2.5 1.5 2.5 1.5 2.0 2.5 1.5 2.0 2.0 2.0 | 2.0875c |
| (Pm line) | | 1.5 3.0 1.5 2.0 2.5 2.0 2.0 2.5 1.0 2.0 | |
| | II | 2.0 2.5 2.5 1.5 2.0 1.5 2.5 2.5 3.0 1.5 | |
| | | 1.5 2.0 2.5 2.5 2.5 2.0 1.5 2.0 2.5 2.5 | |
| H7BFlat-1 × HKN89-3R-1(op)-1-1 | I | 1.5 0.5 1.0 0.0 0.5 0.0 0.5 1.0 0.0 0.0 | 0.6375d |
| | | 0.5 2.0 1.0 1.5 0.0 0.5 0.0 1.0 0.0 0.5 | |
| | II | 1.0 0.0 0.0 2.0 2.0 0.0 0.0 0.0 0.5 1.0 | |
| | | 1.0 0.0 2.0 1.5 0.0 0.0 1.0 1.0 0.5 0.0 | |
| H7BFlat-1 × MlnF411 | I | 0.0 0.5 0.5 0.5 1.0 0.0 0.5 0.0 0.5 0.0 | 0.3500e |
| | | 0.5 1.0 0.0 0.5 0.0 0.0 1.0 0.0 0.0 0.5 | |
| | II | 0.5 0.0 1.0 0.5 0.5 0.0 0.5 0.0 0.5 0.0 | |
| | | 1.0 0.0 0.0 0.5 0.0 0.0 0.5 0.0 0.5 1.0 | |
| H7BFlat-1 × MtlnF42-1 | I | 0.5 0.5 0.0 0.0 0.5 0.0 0.5 0.0 0.0 0.5 | 0.3250e |
| | | 0.5 0.0 1.0 0.5 0.0 0.5 0.0 1.0 0.0 0.5 | |
| | II | 0.0 0.0 0.5 0.5 0.5 0.0 0.0 1.0 0.5 0.0 | |
| | | 0.5 0.0 0.0 0.5 1.0 0.5 0.0 0.5 0.5 0.0 | |
| H7BFlat-1 | I | 0.0 0.5 0.5 0.0 0.5 1.0 0.0 0.0 0.5 0.0 | 0.2875e |
| (Pm2 line) | | 0.5 0.0 0.5 0.5 0.0 0.0 0.0 0.5 0.5 0.0 | |
| | II | 0.5 0.0 1.0 0.0 0.5 0.5 0.5 0.0 0.5 0.0 | |
| | | 0.5 0.0 0.0 0.5 0.0 0.0 1.0 0.5 0.0 0.0 | |

TABLE 4
Inheritance Of Pm2 And Pm In F₂ Populations In 2009
PMR Segregation In Three F₂ Populations

| | | | Chi-square^{z} |
|---|---|---|---|
| | Pm2Pm2 | PmPm | χ²(3:1) |
| F₂ populations in greenhouse | | | |
| (H7BFlat-1 × MltnF42-1)F₂ | 67 | 27 | 0.6950 |
| (H7BFlat-1 × MlnF411)F₂ | 64 | 25 | 0.4532 |
| (H7BFlat-1 × HKN89-3R- | 70 | 29 | 0.9731 |
| 1(op)-1-1)F₂ | | | |
| F₂ populations in Field | | | |
| (H7BFlat-1 × MltnF42-1)F₂ | 20 | 10 | 1.1111 |
| (H7BFlat-1 × MlnF411)F₂ | 21 | 11 | 1.5000 |
| (H7BFlat-1 × HKN89-3R- | 18 | 10 | 1.7143 |
| 1(op)-1-1)F₂ | | | |

### Example 4

### Assessing the Level of Powdery Mildew Resistance Conferred by the Mutant Allele Pm2 of the Present Invention in Hybrid Form Comparing with Commercial Varieties Having Pm

2005 Trial Using Drip Irrigation:

To assess the powdery mildew resistance level conferred by the mutant allele Pm2 of the present invention in hybrid form, three selected commercially available powdery mildew susceptible pumpkin lines BSP1, GDF41, and HDD31 were crossed with the powdery mildew resistant line H7B-1Lop-1-1-1 which contains the mutant allele Pm2 of the present invention, respectively, and formed three hybrids, HSR4706, HSR4707 and HSR4709 in 2004. These hybrids each contain the mutant allele Pm2 of the present invention and were tested for powdery mildew resistance successively in experimental trials during the summers of 2005 and 2006. The level of powdery mildew on plants was divided into six scores or fractions thereof consisting of 0, 1, 2, 3, 4, 5, in which 0 indicates that the plants were completely resistant to the disease and there were no symptoms of the disease on the plants at all, and a rating of 5 indicates that the plants were 100% infected and susceptible. Increasing one level among the six scores means that the resistance to powdery mildew disease decreased by 20% or, conversely, the susceptibility to the disease increased by 20%. For example, a plant with a score of 1 means that plant showed 20% more disease symptoms than a plant with a score of 0. In another example, a plant with a score of 3 means that plant shows 60% more disease symptoms than a plant with a score of 0. Each score is based on the following: the ratio of infected leaves and stems on a plant, the proportion of infected area on a leaf and a stem, and the colony counts out of the total in the infected area. To facilitate the difference among the powdery mildew resistance levels for analysis, a significance test reciprocal transformation was applied during the mean separation process.

The first powdery mildew resistance pumpkin hybrid trial was conducted in the summer of 2005. The experiment was designed to determine whether the new powdery mildew resistance characteristic conferred by the mutant allele Pm2 of the present invention in hybrids is superior to that of what is available in commercial pumpkin varieties. The new hybrids containing the mutant allele Pm2 were HSR4706, HSR4707, and HSR4709. These hybrids were produced from the cross between the homozygous PMR line H7B-1Lop-1-1-1, which contained the mutant allele Pm2 of the present invention, and a commercially available powdery mildew susceptible male. Three commercial varieties, Merlin, Magic Lantern and Spartan, which were considered the best commercially available powdery mildew resistant varieties at that time, were used for comparison. A completely randomized experimental design with two replications was used for each treatment and each treatment consisted of 60 plants grown 36 inches apart within a row covered with plastic mulch and 80 inches between the rows. An underground drip irrigation system accompanying a routine fertilizer supply was used. The trial time period started with direct seeding on May 15, 2005 and was kept as long as possible to allow for a full expression of powdery mildew resistance and plant growth potential in all the tested varieties.

In Table 5, the results of the powdery mildew resistance trial for 2005 are shown. Column 1 shows the variety, column 2 shows the replication, and column 3 shows the mean powdery mildew resistance reading based on the scores of 30 plants per replication. Means were transformed and analyzed using an F test on the transformed mean of the average powdery mildew resistance reading. Means (analyzed as transformed means) bearing the same letters indicate those means that were not significantly different at the 1% level.

As shown in Table 5, the three pumpkin varieties containing the mutant allele Pm2 of the present invention had mean scores of about 1.3 while the three commercially available pumpkin varieties had mean scores of about 3.1. These results indicate that the pumpkin varieties containing the mutant allele Pm2 were highly resistant to powdery mildew, whereas the commercial pumpkin varieties were only slightly to moderately resistant.

TABLE 5
PMR Scores

| Variety | Rep | Mean |
|---|---|---|
| HSR4706 | I | 1.2833 |
| | II | 1.3167 |
| | Overall | 1.3000a |
| HSR4707 | I | 1.3667 |
| | II | 1.2833 |
| | Overall | 1.3334a |
| HSR4709 | I | 1.3333 |
| | II | 1.3500 |
| | Overall | 1.3417a |
| Spartan | I | 2.9667 |
| | II | 3.0333 |
| | Overall | 3.0000b |
| Magic Lantern | I | 3.1333 |
| | II | 3.1500 |
| | Overall | 3.1417b |
| Merlin | I | 3.1500 |
| | II | 3.3000 |
| | Overall | 3.225b |

2006 Trial Using a Furrowed Irrigation System:

The second pumpkin trial for powdery mildew resistance was carried out in the summer of 2006 and had the same experimental design and field lay-out as in the previous 2005 trial. This time, there was no plastic mulch and no drip system was used. Instead, furrow irrigation was used for the trial so that it could make the ground surface as moist as possible to cause the occurrence of powdery mildew.

The level of powdery mildew on plants was divided into six scores or fractions thereof consisting of 0, 1, 2, 3, 4, 5, in which 0 indicates that the plants were completely resistant to the disease and there were no symptoms of the disease on the plants at all, and a rating of 5 indicates that the plants were 100% infected. Increasing one level among the six scores, means that the resistance to the disease decreased by 20% or, conversely, the susceptibility to the disease increased by 20%. For example, a plant with a score of 1 means that plant showed 20% more disease symptoms than a plant with a score of 0. In another example, a plant with a score of 3 means that plant shows 60% more disease symptoms than a plant with a score of 0. Each score is based on the following: the ratio of infected leaves and stems on a plant, the proportion of infected area on a leaf and a stem, and the colony counts out of the total in the infected area. To better understand the difference among the PMR levels, a significance test reciprocal transformation was applied during the mean separation process.

In Table 6, the results of the powdery mildew resistance trial for 2006 are shown. Column 1 shows the variety, column 2 shows the replication, and column 3 shows the mean powdery mildew resistance reading based on the scores of 30 plants per replication. Means were transformed and analyzed using an F test on the transformed mean of the average powdery mildew resistance reading. Means (analyzed as transformed means) bearing the same letters indicate those means that were not significantly different at the 1% level.

As shown in Table 6, the three varieties containing the mutant allele Pm2 had mean powdery mildew resistance scores of about 1.4 while the commercially available pumpkin varieties had mean scores of about 3.4. These results indicate that the pumpkin varieties containing the mutant allele Pm2 were highly resistant to powdery mildew, whereas the commercial pumpkin varieties were moderately to slightly resistant. These scores also show that the mutant allele Pm2 continued to provide the pumpkin plants containing the allele with powdery mildew resistance under increased powdery mildew pressure, whereas the commercially available pumpkin varieties were more heavily affected by the increased powdery mildew presence.

TABLE 6

| Variety | Rep | Mean |
|---|---|---|
| HSR4706 | I | 1.3833 |
| | II | 1.4167 |
| | Overall | 1.3917a |
| HSR4707 | I | 1.4333 |
| | II | 1.3833 |
| | Overall | 1.4083a |
| HSR4709 | I | 1.4167 |
| | II | 1.4500 |
| | Overall | 1.4250a |
| Spartan | I | 3.2500 |
| | II | 3.2667 |
| | Overall | 3.2583b |
| Magic Lantern | I | 3.3667 |
| | II | 3.3833 |
| | Overall | 3.3750b |
| Merlin | I | 3.3833 |
| | II | 3.4500 |
| | Overall | 3.4167b |

The powdery mildew screening tests showed that all of the pumpkin plants containing the mutant allele Pm2 of the present invention unexpectedly had much greater resistance to powdery mildew than any of the commercial pumpkin varieties in the two preceding trials from 2005 and 2006. Unexpectedly, when the commercial pumpkin varieties began to die by the beginning of September because of powdery mildew infection, all of the pumpkin plants containing the mutant allele Pm2 of the present invention continued to grow and bear fruit until the first killing frost occurred. The phenotypic expression of the new powdery mildew mutant allele Pm2 in the new pumpkin hybrids was unexpectedly found to be more than two times greater than the powdery mildew resistance displayed by the commercial varieties based on the differences between the means in Tables 5 and 6. Statistical results in Tables 5 and 6 show that the powdery mildew resistance levels between the new pumpkin hybrids containing the mutant allele Pm2 of the present invention and the powdery mildew resistance levels of the commercial pumpkin varieties were significantly different (F_{0.01}). The powdery mildew resistance exhibited by the new varieties, which contain the mutant allele Pm2 of the present invention, is significantly better than the powdery mildew resistance exhibited by currently available commercial varieties.

2007 Trial for Powdery Mildew Resistance (Trial 5):

The third pumpkin trial for powdery mildew resistance was carried out in the summer of 2007 and the data was collected on Aug. 15, 2007 and Sep. 15, 2007. To determine the differences in powdery mildew resistance (PMR) between pumpkin plants of the present invention containing the Pm2 allele and commercial pumpkin varieties, the trial was conducted in southern Colorado with a randomized block layout, which adopted two replications and used leaves, petioles and stems as three treatments for the objective of investigating the degree of powdery mildew infection. Due to the continuous dying-off of the commercial plants Merlin, Magic Lantern and Spartan during the growing season, the missed plants in each plot were left out and only the truncated data that was collected on Aug. 15, 2007 (Table 7) from the first six survived plants was used in the statistical analyses. The data (Table 8) from Sep. 15, 2007 was not statistically analyzed because most of the observed plants of some commercial varieties were lost due (died) to the powdery mildew infection during the growing season and is just used for general information herein.

The level of powdery mildew on plants was counted as the mean number powdery mildew colonies found per square inch on a leaf, petiole or stem of the pumpkin plant. The plants were about 90 days old.

Table 7 shows the data of the powdery mildew resistance trial collected from the pumpkin varieties of the present invention, HSR4706, HSR4707, HSR4709 HSR4710, HSR4711 and HSR4720, with commercial pumpkin varieties Merlin, Magic Lantern and Spartan on Aug. 15, 2007. Table 7 consists of two blocks (I and II). Column 1 shows the variety, column 2 shows the replication, column 3 shows the observed organs as three treatments (leaf, stem, and petiole), and column 4 shows the mean number of colonies of powdery mildew per square inch on a particular plant organ (leaf, petiole, or stem), which is derived from 10 plants listed in the 10 sub-columns. Each entry in the 10 sub-columns represents the mean number of colony counts on different locations of observed plant parts (the leaf, petiole, or stem) from each individual plant. Entries that do not contain a number (blank cells) mean that the plants did not survive (died) during the trial.

TABLE 7

| | | | Mean Colonies/inch² | | | | | | | | | |
|---|---|---|---|---|---|---|---|---|---|---|---|---|
| Trial | | Observed | Group (weeks after planting) | | | | | | | | | |
| Entries | Reps | Organs | 1 | 2 | 3 | 4 | 5 | 6 | 7 | 8 | 9 | 10 |

Block I

| | | | | | | | | | | | | |
|---|---|---|---|---|---|---|---|---|---|---|---|---|
| HSR4710 | 1 | Leaf | 0 | 0 | 0 | 0 | 0 | 0 | 1 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| | 2 | Leaf | 0 | 1 | 0 | 0 | 1 | 0 | 0 | 0 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| Merlin | 1 | Leaf | 4 | 6 | 5 | 5 | 5 | 6 | 5 | | | |
| | | Petiole | 2 | 2 | 4 | 4 | 3 | 3 | 4 | | | |
| | | Stem | 1 | 1 | 3 | 2 | 3 | 3 | 2 | | | |
| | 2 | Leaf | 4 | 5 | 6 | 4 | 5 | 6 | | | | |
| | | Petiole | 1 | 2 | 4 | 4 | 3 | 3 | | | | |
| | | Stem | 3 | 2 | 2 | 3 | 3 | 3 | | | | |
| HSR4706 | 1 | Leaf | 0 | 0 | 0 | 0 | 1 | 0 | 0 | 0 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| | 2 | Leaf | 0 | 0 | 2 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| HSR4720 | 1 | Leaf | 0 | 0 | 0 | 0 | 0 | 0 | 0 | 0 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| | 2 | Leaf | 0 | 0 | 0 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Magic | 1 | Leaf | 2 | 2 | 3 | 4 | 4 | 4 | 3 | | | |
| Lantern | | Petiole | 1 | 2 | 2 | 1 | 3 | 2 | 2 | | | |
| | | Stem | 1 | 1 | 1 | 2 | 2 | 2 | 1 | | | |
| | 2 | Leaf | 4 | 3 | 3 | 5 | 4 | 4 | | | | |
| | | Petiole | 2 | 1 | 1 | 3 | 2 | 2 | | | | |
| | | Stem | 2 | 1 | 1 | 2 | 2 | 2 | | | | |
| HSR4707 | 1 | Leaf | 0 | 0 | 0 | 0 | 1 | 0 | 0 | 0 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| | 2 | Leaf | 1 | 0 | 0 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Spartan | 1 | Leaf | 3 | 3 | 2 | 4 | 4 | 3 | | | | |
| | | Petiole | 3 | 1 | 1 | 2 | 3 | 2 | | | | |
| | | Stem | 1 | 2 | 2 | 2 | 3 | 2 | | | | |
| | 2 | Leaf | 3 | 3 | 3 | 4 | 3 | 3 | | | | |
| | | Petiole | 3 | 1 | 2 | 3 | 3 | 2 | | | | |
| | | Stem | 2 | 3 | 2 | 2 | 2 | 2 | | | | |
| HSR4709 | 1 | Leaf | 0 | 0 | 0 | 0 | 0 | 0 | 0 | 1 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 0 | 1 | 0 | 0 | 0 | 0 | 0 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| HSR4711 | 1 | Leaf | 0 | 0 | 0 | 1 | 0 | 0 | 0 | 1 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 0 | 0 | 0 | 0 | 0 | 1 | 0 | 0 | 1 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |

Block II

| | | | | | | | | | | | | |
|---|---|---|---|---|---|---|---|---|---|---|---|---|
| HSR4709 | 1 | Leaf | 0 | 0 | 0 | 2 | 0 | 0 | 0 | 0 | 0 | 1 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 0 | 1 | 0 | 1 | 0 | 0 | | | |
| | | Petiole | 0 | 0 | 0 | 0 | 0 | 0 | 0 | | | |
| | | Stem | 0 | 0 | 0 | 0 | 0 | 0 | 0 | | | |
| HSR4710 | 1 | Leaf | 0 | 1 | 0 | 0 | 0 | 0 | 0 | 1 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 1 | 0 | 0 | 0 | 0 | 0 | 0 | 0 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| Magic | 1 | Leaf | 4 | 4 | 4 | 2 | 3 | 3 | 3 | | | |
| Lantern | | Petiole | 3 | 3 | 3 | 3 | 2 | 3 | 3 | | | |
| | | Stem | 2 | 2 | 3 | 3 | 3 | 2 | 3 | | | |
| | 2 | Leaf | 4 | 4 | 3 | 3 | 4 | 4 | 3 | | | |
| | | Petiole | 4 | 3 | 3 | 3 | 3 | 3 | 2 | | | |
| | | Stem | 3 | 3 | 3 | 3 | 2 | 3 | 3 | | | |
| HSR4707 | 1 | Leaf | 0 | 0 | 0 | 0 | 1 | 0 | 0 | 0 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 1 | 0 | 2 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Spartan | 1 | Leaf | 3 | 3 | 3 | 4 | 3 | 3 | 4 | | | |
| | | Petiole | 3 | 3 | 2 | 3 | 3 | 3 | 3 | | | |
| | | Stem | 2 | 3 | 2 | 3 | 2 | 2 | 3 | | | |
| | 2 | Leaf | 2 | 2 | 4 | 3 | 2 | 3 | 4 | | | |
| | | Petiole | 3 | 2 | 2 | 2 | 2 | 3 | 2 | | | |
| | | Stem | 2 | 2 | 2 | 2 | 3 | 2 | 3 | | | |
| HSR4720 | 1 | Leaf | 0 | 0 | 0 | 0 | 0 | 0 | 0 | 0 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 0 | 0 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| HSR4706 | 1 | Leaf | 0 | 1 | 0 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| | 2 | Leaf | 0 | 1 | 1 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Merlin | 1 | Leaf | 3 | 3 | 3 | 4 | 4 | 3 | | | | |
| | | Petiole | 3 | 3 | 2 | 4 | 3 | 2 | | | | |
| | | Stem | 2 | 3 | 3 | 3 | 2 | 3 | | | | |
| | 2 | Leaf | 4 | 3 | 4 | 4 | 3 | 4 | | | | |
| | | Petiole | 3 | 3 | 3 | 4 | 4 | 3 | | | | |
| | | Stem | 4 | 3 | 3 | 3 | 2 | 3 | | | | |
| HSR4711 | 1 | Leaf | 0 | 0 | 0 | 0 | 0 | 0 | 0 | 2 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| | 2 | Leaf | 0 | 0 | 0 | 0 | 1 | 0 | 0 | 0 | 0 | 1 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |

TABLE 8

| | | | Mean Colonies/inch² | | | | | | | | | |
|---|---|---|---|---|---|---|---|---|---|---|---|---|
| Trial | | Observed | Group (weeks after planting) | | | | | | | | | |
| Entries | Reps | Organs | 1 | 2 | 3 | 4 | 5 | 6 | 7 | 8 | 9 | 10 |

Block I

| | | | | | | | | | | | | |
|---|---|---|---|---|---|---|---|---|---|---|---|---|
| HSR4710 | 1 | Leaf | 0 | 0 | 1 | 0 | 0 | 0 | 1 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| | 2 | Leaf | 0 | 1 | 0 | 0 | 1 | 0 | 0 | | | |
| | | Petiole | 0 | 0 | 0 | 0 | 0 | 0 | 0 | | | |
| | | Stem | 0 | 0 | 0 | 0 | 0 | 0 | 0 | | | |
| Merlin | 1 | Leaf | 5 | 6 | | | | | | | | |
| | | Petiole | 2 | 3 | | | | | | | | |
| | | Stem | 1 | 2 | | | | | | | | |
| Merlin | 1 | Leaf | 1 | 0 | 0 | 0 | 1 | 0 | 0 | 0 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| | 2 | Leaf | 5 | 5 | | | | | | | | |
| | | Petiole | 2 | 3 | | | | | | | | |
| | | Stem | 3 | 3 | | | | | | | | |
| HSR4706 | 1 | Leaf | 0 | 0 | 0 | 0 | 1 | 0 | 0 | 0 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| | 2 | Leaf | 0 | 0 | 2 | 0 | 0 | 0 | 0 | 0 | 1 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| HSR4720 | 1 | Leaf | 0 | 0 | 0 | 0 | 0 | 0 | 0 | 0 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| | 2 | Leaf | 0 | 0 | 0 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Magic Lantern | 1 | Leaf | 4 | 2 | 3 | 4 | 4 | 4 | | | | |
| | | Petiole | 2 | 3 | 2 | 2 | 3 | 3 | | | | |
| | | Stem | 1 | 2 | 1 | 3 | 2 | 3 | | | | |
| | 2 | Leaf | 4 | 4 | 4 | 5 | | | | | | |
| | | Petiole | 3 | 2 | 2 | 4 | | | | | | |
| | | Stem | 2 | 2 | 2 | 3 | | | | | | |
| HSR4707 | 2 | Leaf | 1 | 0 | 0 | 0 | 0 | 0 | 0 | 0 | 1 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Spartan | 1 | Leaf | 3 | 4 | 3 | | | | | | | |
| | | Petiole | 3 | 1 | 2 | | | | | | | |
| | | Stem | 3 | 2 | 2 | | | | | | | |
| | 2 | Leaf | 3 | 4 | 3 | | | | | | | |
| | | Petiole | 3 | 2 | 3 | | | | | | | |
| | | Stem | 2 | 3 | 3 | | | | | | | |
| HSR4709 | 1 | Leaf | 0 | 0 | 1 | 0 | 0 | 0 | 0 | 1 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 0 | 1 | 0 | 0 | 0 | 0 | 2 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| HSR4711 | 1 | Leaf | 1 | 0 | 0 | 1 | 0 | 0 | 0 | 1 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 1 | 0 | 0 | 0 | 0 | 1 | 0 | 0 | 1 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |

Block II

| | | | | | | | | | | | | |
|---|---|---|---|---|---|---|---|---|---|---|---|---|
| HSR4709 | 1 | Leaf | 1 | 0 | 0 | 2 | 0 | 0 | 0 | 0 | 0 | 1 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 0 | 1 | 0 | 1 | 0 | 0 | | | |
| | | Petiole | 0 | 0 | 0 | 0 | 0 | 0 | 0 | | | |
| | | Stem | 0 | 0 | 0 | 0 | 0 | 0 | 0 | | | |
| HSR4710 | 1 | Leaf | 1 | 0 | 0 | 0 | 0 | 0 | 1 | 0 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| HSR4710 | 2 | Leaf | 0 | 1 | 0 | 0 | 0 | 0 | 0 | 1 | | |
| | | Petiole | 0 | 0 | 0 | 0 | 0 | 0 | 0 | 0 | | |
| | | Stem | 0 | 0 | 0 | 0 | 0 | 0 | 0 | 0 | | |
| Magic Lantern | 1 | Leaf | 4 | 5 | 4 | | | | | | | |
| | | Petiole | 3 | 3 | 3 | | | | | | | |
| | | Stem | 2 | 3 | 3 | | | | | | | |
| | 2 | Leaf | 4 | 4 | 3 | | | | | | | |
| | | Petiole | 4 | 5 | 3 | | | | | | | |
| | | Stem | 3 | 3 | 4 | | | | | | | |
| HSR4707 | 1 | Leaf | 0 | 0 | 0 | 0 | 1 | 0 | 0 | 0 | 1 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| | 2 | Leaf | 1 | 0 | 2 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Spartan | 1 | Leaf | 3 | 4 | 4 | | | | | | | |
| | | Petiole | 3 | 4 | 2 | | | | | | | |
| | | Stem | 2 | 3 | 3 | | | | | | | |
| | 2 | Leaf | 4 | 3 | 4 | 3 | | | | | | |
| | | Petiole | 3 | 2 | 3 | 2 | | | | | | |
| | | Stem | 2 | 3 | 2 | 3 | | | | | | |
| HSR4720 | 1 | Leaf | 0 | 0 | 0 | 0 | 0 | 0 | 0 | 0 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| | 2 | Leaf | 0 | 0 | 0 | 0 | 0 | 0 | 0 | 0 | 0 | 0 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |
| HSR4706 | 1 | Leaf | 0 | 1 | 1 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| | 2 | Leaf | 0 | 1 | 1 | 0 | 0 | 0 | 0 | 0 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| Merlin | 1 | Leaf | 3 | 4 | | | | | | | | |
| | | Petiole | 4 | 3 | | | | | | | | |
| | | Stem | 2 | 3 | | | | | | | | |
| Merlin | 2 | Leaf | 4 | 4 | | | | | | | | |
| | | Petiole | 3 | 3 | | | | | | | | |
| | | Stem | 4 | 3 | | | | | | | | |
| HSR4711 | 1 | Leaf | 0 | 1 | 0 | 0 | 0 | 0 | 0 | 2 | 0 | |
| | | Petiole | 0 | 0 | 0 | 0 | 0 | 0 | 0 | 0 | 0 | |
| | | Stem | 0 | 0 | 0 | 0 | 0 | 0 | 0 | 0 | 0 | |
| | 2 | Leaf | 0 | 1 | 0 | 0 | 1 | 0 | 0 | 0 | 0 | 1 |
| | | Petiole | 0 | 0 | 0 | 0 | 0 | 0 | 0 | 0 | 0 | 0 |
| | | Stem | 0 | 0 | 0 | 0 | 0 | 0 | 0 | 0 | 0 | 0 |

Statistical analysis using ANOVA of the data contained in Table 7 showed that the mean number of powdery mildew colonies on three parts (treatments) of plants were significantly different between the varieties containing the mutant allele Pm2 and the commercial varieties Merlin, Magic Lantern and Spartan (F=59.57, P<0.0001) (Table 7). For example, HSR4706 and HSR4707 both have a mean of 0.083 powdery mildew colonies, HSR4709 has a mean of about 0.069 powdery mildew colonies, HSR4710 has a mean of about 0.069 powdery mildew colonies, HSR4711 has a mean of about 0.042 powdery mildew colonies and HSR4720 has a mean of 0 powdery mildew colonies, while Magic Lantern has a mean of about 2.681 powdery mildew colonies, Merlin has a mean of about 3.306 powdery mildew colonies and Spartan has a mean of about 2.528 powdery mildew colonies. Using an F-test, when the number of powdery mildew colonies found per square inch per plant of individual varieties HSR4710, HSR4706, HSR4707, HSR4709HSR4710, HSR4720 is compared individually with the number of powdery mildew colonies found on Merlin, Magic Lantern or Spartan, results indicate that each of the varieties of the present invention (HSR4710, HSR4706, HSR4707, HSR4709 HSR4710, HSR4720) have statistically significantly fewer powdery mildew colonies, (P<0.0001) than either Merlin, Magic Lantern or Spartan. The results indicate that the pumpkin varieties containing the mutant allele Pm2 of the present invention were at the same statistically significant level of powdery mildew resistance comparing that the commercial variety Merlin was significantly susceptible than Spartan and Magic Lantern. Overall, plants containing the Pm2 mutant allele of the present invention are significantly more resistant to powdery mildew than commercial varieties not containing the mutant allele Pm2 (Merlin, Magic Lantern and Spartan) (α=0.05).

Table 9 shows the mean separation or paired comparison for colony counts for the pumpkin varieties containing the mutant allele Pm2 of the present invention with commercial pumpkin varieties Merlin, Magic Lantern and Spartan. “z” means with the same letter are not significantly different at 0.05 level. Magic Lantern, Spartan and Merlin are commercial pumpkin varieties used as comparisons, which do not carry the mutant PMR allele. “y” means the treatments consist of leaves, stems and petioles.

TABLE 9

| Varieties | Blocks | Replications | Treatments^{y} | Mean^{z} |
|---|---|---|---|---|
| Merlin | 2 | 2 | 3 | 3.306 a |
| Magic Lantern | 2 | 2 | 3 | 2.681 b |
| Spartan | 2 | 2 | 3 | 2.528 b |
| HSR4707 | 2 | 2 | 3 | 0.083 c |
| HSR4706 | 2 | 2 | 3 | 0.083 c |
| HSR4710 | 2 | 2 | 3 | 0.069 c |
| HSR4709 | 2 | 2 | 3 | 0.069 c |
| HSR4711 | 2 | 2 | 3 | 0.042 c |
| HSR4720 | 2 | 2 | 3 | 0.000 c |

2007 Trial for Powdery Mildew Resistance (Trial 6):

The data in Table 8 shows that the pumpkin varieties of the present invention, HSR4706, HSR4707, HSR4709 HSR4710, HSR4711 and HSR4720 grew vigorously during the entire season while the plants of commercial pumpkin varieties Merlin, Magic Lantern and Spartan continuously died out due to their susceptibility to the powdery mildew disease. Entries that do not contain a number (blank cells) mean that the plants died during the trial. When the plants were about 120 days old, the observed plants of Merlin, Spartan and Magic Lantern were about 80%, 70% and 60% died, respectively. It can be concluded that the pumpkin varieties containing mutant allele Pm2 of the present invention have a significant higher surviving rate and have a more consistent resistance to powdery mildew than the commercial products under a high powdery mildew pressure.

An unexpected and significant difference of the mutant allele Pm2 versus Pm is that the lines or crosses carrying Pm2 always express Pm2 in a constant dominant manner at the seedling stage and at maturity and have a consistent resistance to powdery mildew during the complete growing season. Tables 5 and 6 in the application show the phenotypic expression of the commercial varieties with Pm, Merlin, Magic Lantern and Spartan, analogue to that of Contin's 1978 experimental materials; susceptibility of which became higher so that many plants died from the powdery mildew infection as they were getting older. However, all crosses with the mutant allele Pm2 unexpectedly had a persistent and consistent powdery mildew resistance in the whole growing season and had a second fruit setting when all plants of commercial varieties with Pm were stopped from growing or dying from the disease. Similar results have been proven in many trials during the last four years. Hollar PMR pumpkins Camaro, HSR4710 and HSR4721 had the lowest powdery mildew infection rate in the 2008 trial of Central Kentucky (Timothy Coolong and Kenneth Seebold, Evaluation of Powdery Mildew Tolerance in Pumpkin in Central Kentucky); the first two varieties, Hollar PMR pumpkins Camaro and HSR4710, also had the lowest amount of powdery mildew at 1% during the whole growing season (Elizabeth T. Maynard, Pumpkin Variety performance with and without treatment for powdery mildew in Northern Indiana, 2008). The fruit number was significantly higher for Hollar PMR pumpkins Camaro, HSR4710 and HSR4721 than the other tested varieties due to their PMR.

Fruit and Yield Trial Comparisons Under Powdery Mildew Pressure:

The yield data in Table 10 were taken from the survived plants in each plot across two replications and two blocks of a completely randomized-block-designed trial on Sep. 15, 2007. The plot yields were calculated based on marketable fruit number and their average weight after all rotten fruit were discarded. From the total number and the number of survived plants in all the plots, it shows that commercial pumpkin varieties Merlin, Magic Lantern and Spartan continually died-off during the growing season and lost about 50-80% of their yield at the end of the season due to the effects of powdery mildew. Column 1 in the table shows the variety, column two shows the replication number, column 3 shows the number of plants, column 4 shows the number of plants that survived, column 5 shows the fruit diameter in inches, column 6 shows the fruit height in inches, column 7 shows the total number of fruit, column 8 shows the number of rotten fruit, column 9 shows the number of marketable fruit, column 10 shows the average fruit weight in kilograms and column 11 shows the plot yield in kilograms.

TABLE 10

| | | | | | | | | | | |
|---|---|---|---|---|---|---|---|---|---|---|
| | | | # of | Fruit | Fruit | | | | Avg. | |
| Trial Entries | | # of | Plants | Diameter | Height | Total | Rotten | Marketable | Fruit wt | Plot yield |
| (Variety) | Rep | Plants | survived | (Inches) | (Inches) | fruit | fruit | fruit | (kg) | (kg) |

(Block I)

| | | | | | | | | | | |
|---|---|---|---|---|---|---|---|---|---|---|
| HSR4710 | 1 | 9 | 9 | 13.3 | 12.5 | 12 | 0 | 12 | 11.5 | 138 |
| | 2 | 8 | 7 | 13.5 | 12.25 | 12 | 1 | 11 | 12 | 132 |
| Merlin | 1 | 8 | 2 | 10.5 | 9.0 | 8 | 6 | 2 | 6.5 | 13 |
| | 2 | 10 | 3 | 11.0 | 10.5 | 14 | 8 | 6 | 7 | 42 |
| HSR4706 | 1 | 9 | 8 | 12.0 | 13.0 | 16 | 1 | 15 | 11.5 | 172.5 |
| | 2 | 12 | 10 | 11.5 | 12.5 | 25 | 2 | 23 | 11 | 253 |
| HSR4720 | 1 | 8 | 8 | 12.5 | 11.5 | 18 | 0 | 18 | 9.7 | 174.6 |
| | 2 | 10 | 9 | 12.0 | 11.0 | 20 | 3 | 17 | 9.5 | 161.5 |
| Magic Lantern | 1 | 13 | 6 | 13.5 | 11.0 | 18 | 9 | 9 | 10.5 | 94.5 |
| | 2 | 8 | 4 | 13.0 | 11.5 | 17 | 14 | 3 | 9.9 | 29.7 |
| HSR4721 | 1 | 8 | 8 | 11.0 | 9.5 | 22 | 0 | 22 | 6.9 | 151.8 |
| | 2 | 11 | 11 | 10.5 | 9.5 | 29 | 0 | 29 | 6.5 | 188.5 |
| HSR4707 | 1 | 10 | 9 | 10.0 | 11.5 | 21 | 0 | 21 | 8.6 | 180.6 |
| | 2 | 8 | 8 | 10.0 | 10.0 | 19 | 0 | 19 | 7.6 | 144.4 |
| HSR4722 | 1 | 7 | 7 | 11.5 | 10.5 | 18 | 1 | 17 | 8 | 136 |
| | 2 | 10 | 10 | 11.5 | 10.0 | 23 | 0 | 23 | 7.5 | 172.5 |
| Spartan | 1 | 11 | 3 | 9.5 | 9.0 | 20 | 13 | 7 | 6.5 | 45.5 |
| | 2 | 6 | 3 | 10.0 | 9.0 | 7 | 4 | 3 | 7 | 21 |
| HSR4709 | 1 | 10 | 10 | 13.5 | 13.0 | 18 | 0 | 18 | 12 | 216 |
| | 2 | 10 | 10 | 13.5 | 12.5 | 15 | 0 | 15 | 11.5 | 172.5 |
| HSR4711 | 1 | 13 | 12 | 12.0 | 11.25 | 20 | 2 | 18 | 8.6 | 154.8 |
| | 2 | 11 | 10 | 11.5 | 10.0 | 15 | 3 | 12 | 7.3 | 87.6 |

(Block II)

| | | | | | | | | | | |
|---|---|---|---|---|---|---|---|---|---|---|
| HSR4709 | 1 | 7 | 7 | 12.5 | 13.5 | 13 | 2 | 11 | 11 | 121 |
| | 2 | 10 | 10 | 11.0 | 12.5 | 16 | 1 | 15 | 10 | 150 |
| HSR4722 | 1 | 9 | 9 | 10.5 | 9.5 | 18 | 1 | 17 | 6.5 | 110.5 |
| | 2 | 9 | 8 | 11.0 | 10.5 | 18 | 2 | 16 | 7 | 112 |
| HSR4710 | 1 | 11 | 11 | 10.5 | 10.0 | 21 | 0 | 21 | 6.3 | 132.3 |
| | 2 | 12 | 11 | 12.0 | 9.0 | 17 | 0 | 17 | 9.5 | 161.5 |
| Magic Lantern | 1 | 10 | 3 | 13.0d | 12.5 | 20 | 16 | 4 | 10 | 40 |
| | 2 | 11 | 3 | 12.5 | 10.5 | 19 | 16 | 3 | 8.5 | 25.5 |
| HSR4721 | 1 | 10 | 10 | 11.0 | 10.0 | 25 | 0 | 25 | 7 | 175 |
| | 2 | 9 | 9 | 10.5 | 10.0 | 22 | 0 | 22 | 7 | 154 |
| HSR4707 | 1 | 9 | 9 | 12.0 | 11.5 | 17 | 0 | 17 | 11 | 187 |
| | 2 | 10 | 9 | 10.5 | 10.0 | 24 | 2 | 22 | 6.5 | 143 |
| Spartan | 1 | 9 | 4 | 10.5 | 9.0 | 17 | 12 | 5 | 6.7 | 33.5 |
| | 2 | 9 | 3 | 11.0 | 9.5 | 13 | 7 | 6 | 7 | 42 |
| HSR4720 | 1 | 12 | 12 | 9.0 | 10.5 | 25 | 0 | 25 | 7.5 | 187.5 |
| | 2 | 10 | 10 | 11.0 | 12.0 | 23 | 0 | 23 | 10.7 | 246.1 |
| HSR4706 | 1 | 10 | 9 | 12.0 | 12.5 | 21 | 0 | 21 | 8.8 | 184.8 |
| | 2 | 9 | 9 | 11.5 | 12.0 | 20 | 0 | 20 | 8 | 160 |
| Merlin | 1 | 8 | 2 | 10.5 | 9.0 | 12 | 10 | 2 | 6.8 | 13.6 |
| | 2 | 12 | 2 | 10.0 | 8.5 | 19 | 16 | 3 | 6.5 | 19.5 |
| HSR4711 | 1 | 10 | 10 | 10.5 | 12.0 | 16 | 0 | 16 | 9.5 | 152 |
| | 2 | 9 | 9 | 9.0 | 12.0 | 14 | 1 | 13 | 9 | 117 |

Table 11 below shows the mean separation or multiple paired comparison for yield for the pumpkin varieties containing the mutant allele Pm2 of the present invention with commercial pumpkin varieties Merlin, Magic Lantern and Spartan. “z” means with the same letter are not significantly different at 0.05 level. Magic Lantern, Spartan and Merlin are commercial pumpkin varieties used as comparisons, which do not carry the mutant Pm2 allele.

By multiple paired comparisons of yields among pumpkins containing the mutant allele Pm2 of the present invention and commercial pumpkin varieties Merlin, Magic Lantern and Spartan, it was found that the yields of the pumpkins containing the Pm2 mutant allele of the present invention were statistically significantly higher than the commercial pumpkin varieties Magic, Lantern and Spartan and of Merlin at 0.05 level. Although pumpkins of the present invention HSR4706, HSR4720 have a significantly higher yield than HSR4721, HSR4709 and 4707 which have a significantly higher yield than HSR4710, HSR4722 and HSR4711, the commercial varieties Magic Lantern, Spartan and Merlin are about 70% lower in their yields due to the powdery mildew infection.

TABLE 11

| Varieties | Blocks | Replications | Mean (Kg)^{z} |
|---|---|---|---|
| HSR4706 | 2 | 2 | 192.47 a |
| HSR4720 | 2 | 2 | 192.43 a |
| HSR4721 | 2 | 2 | 167.32 ab |
| HSR4709 | 2 | 2 | 164.87 ab |
| HSR4707 | 2 | 2 | 163.75 ab |
| HSR4710 | 2 | 2 | 140.95 b |
| HSR4722 | 2 | 2 | 132.75 b |
| HSR4711 | 2 | 2 | 127.85 b |
| Magic Lantern | 2 | 2 | 47.42 c |
| Spartan | 2 | 2 | 35.50 c |
| Merlin | 2 | 2 | 22.02 c |

### Example 5

### Describing Inbred Pumpkin HSPMR7B1 Containing Mutant Allele Pm2 of the Present Invention

Inbred pumpkin HSPMR7B1 contains mutant allele Pm2 and has the following morphologic and other characteristics (based primarily on data collected at Rocky Ford, Colo.).

### HSPMR7B1 Variety Description

Genus: Cucurbita

Species: pepo

Type: Pumpkin

Cotyledons:
Length: 8.0 cm
Width: 4.5 cm
Veining: Obscure
Color: Medium green

Plant: Reduced vine
Prickly

Leaves:
Shape: Heart-shaped; shallow lobe
Margin: Dentate
Length: 40.0 cm
Width: 34.0 cm
Upper-surface: Bristled
Ventral surface: Bristled
Color: Medium green; not blotched

Petiole:
Length: 41.0 cm

Flower (staminate):
Sepals:
   Length: 3.0 cm
   Width: 0.15 cm
Pedicel:
   Length: 30.0 cm
Color: Bright yellow

Fruit (at market maturity):
Height: 30.0 cm
Width: 31.0 cm
Apex: Flat-rounded
Base: Flat-rounded
Ribs: None
Rib furrows: Inconspicuous
Fruit surface: Smooth
Warts: None
Blossom scar button: Slightly depressed
Rind: Medium hard
   Thickness: 0.2 cm
   Color pattern: Regular; orange
Flesh:
   Thickness:
      Blossom end: 2.7 cm
      Medial: 3.0 cm
      Stem end: 3.5 cm
   Texture: Stringy; firm; moist
   Color: Yellow-orange
Seed cavity (sectioned apex to base):
   Length: 24.0 cm
   Width: 25.0 cm
   Location: Conforms to fruit shape
   Placental tissue: Abundant
   Center core: Inconspicuous
Fruit stalks:
   Length: 11.0 cm
   Diameter: 3.5 cm
   Texture: Medium hard
   Furrows: Medium deep
   Surface: Slightly spiny
   Attachment end: Expanded
   Detaches: With difficulty
   Color: Medium green

Seeds:
Length: 2.0 cm
Width: 1.1 cm
Thickness: 0.2 cm
Face surface: Smooth
Color: Cream
Margin: Wedge-like
Separation from pulp: Easy
Weight of 100 seeds: 18.0 gm
Powdery mildew resistance rating: Resistant

### Example 6

### Determining Genetic Similarity Between the Mutant Allele Pm2 of the Present Invention and that of Three Commercially Available Pumpkin Varieties

The genetic similarity between the mutant allele Pm2 of the present invention and that of three commercially available pumpkin varieties was determined. DNA-based SSR, ISSR and RAPD markers were used to compare the genetic relationships of four pumpkin hybrids in April of 2006. The four pumpkin hybrids were Merlin, Spartan, Frosty, and HSR4709. Merlin and Spartan are commercially available pumpkin hybrids that display a lower level of powdery mildew resistance, while Frosty is a commercially available pumpkin hybrid which is powdery mildew susceptible and was used as a control. HSR4709 is a pumpkin hybrid containing mutant allele Pm2 of the present invention. Seeds of the four hybrids were planted and germinated in a greenhouse for the purpose of leaf sampling for DNA extraction. Approximately five weeks post germination date about 40 young leaves from each variety were pooled and placed into sterile 1.5 ml microcentrifuge tubes. Tubes containing the leaf samples were freeze-dried overnight with caps open. Genomic DNA was isolated from each entry using a modified CTAB method, quantified by a DNA Fluorometer (HOEFER DyNA Quant 200, Amersham Pharmacia Biotech, USA) and adjusted to 25 ng/μl in sterile TE buffer.

Three different sets of oligonucleotide primers were used to amplify genomic DNA of each entry with minor modifications. All PCR amplifications were carried out in duplicate for accuracy and amplification consistency. Microsatellite markers designed for C. melo (Chiba et al. 2003, Breeding Science 53:21-27), Inter-simple-sequence-repeat (ISSR) primers of 15-23 nucleotides in length (UBC set #9, Biotechnology Laboratory, University of British Columbia, Vancouver, Canada) and Random Amplified Polymorphic DNA Primers (RAPD) of 10 bases in length (Set #9, Genemed Synthesis, Inc. South San Francisco, Calif., USA) were applied for DNA amplification. Microsatellite (SSR) and ISSRs were amplified as described by Chiba et al. (2003) using 160 mM (NH₄)₂SO₄, 670 mM Tris-HCl (pH 8.8 at 25° C.), 0.1% Tween-20, 2.0 mM MgCl₂, 2.5 mM dNTP, 4 μM primer, 25 ng of genomic DNA per 10 μl of reaction volume and 1.0 U of Biolase DNA Polymerase (Bioline USA Inc., Kenilworth, N.J., USA) for 1 cycle denaturation at 94° C. for 3 minutes, followed by 35 cycles at 94° C. for 60 seconds, 45-60° C. for 60 sec, and 72° C. for 1 minute, and a 5 minute final extension at 72° C. DNA amplification by 10-mer oligonucleotide primers was performed exactly as described above except at an annealing temperature of 35° C. for a total of 40 PCR cycles.

All generated amplicons were separated on 6% polyacrylamide gels. Gels were stained with silver staining method and scored for presence and absence of bands. A locus was considered to be polymorphic if the band was present in one entry and not in the other. The assumption was made that the scored bands were homozygous. Slight distortion of genetic distance could exist by over-estimating genetic similarity with the possibility of the presence of heterozygous bands, which is of greater concern in backcross breeding program than in a database. Monomorphic bands were scored but were not included in any calculations. All of the genotypic data for each entry was scored as a dominant marker.

Coefficients for genetic similarity (GS) between pairs of cultivars were calculated according to Nei and Li (1979):

GS=2Nᵢⱼ/(Nᵢ+Nⱼ)

Where GS equals the similarity coefficient between cultivars i and j, Nᵢⱼ equals the number of common bands present in both i and j cultivars, and Nᵢ and Nⱼ reflect the total of bands detected in cultivars i and j, respectively. GS assessment may attain any value between 0 and 1, where 0 means “no bands in common” and 1 means “patterns are identical.” Consequently, identity of two cultivars will give rise to a GS value of 1, while totally unrelated cultivars will give rise to a GS value of zero. The larger the GS value the more similarities between the entries. It is important to state that a DNA fingerprint of a particular sample is rarely informative on its own. Therefore, as a comparative analysis, the DNA patterns of different samples have to be compared to each other in order to estimate the degree of relatedness. The generated GS values are not absolute and often yield only approximations, becoming intricate to interpret. Such values may be relative to the variability within and between the genetic populations, methodological parameters such DNA marker systems and the statistical methods used for the analysis. For example, a GS value of 0.900 may be interpreted as genetically being the same variety in one species, such as in a highly heterogeneous and obligate open-pollinating crop with an elevated inherent variability within a given variety. In other species, such as in highly homozygous inbred and self-pollinating lines or varieties, a high GS value of 0.980 or 1.00 may be required to demonstrate genetic identity.

Three types of DNA markers were employed in this study, melon microsatellites (SSR), ISSR and RAPD. The genomic DNA of each entry was analyzed in duplicates using 14, 44 and 36 melon microsatellites (SSR), ISSR and RAPD primers, respectively. Repeatable amplifications were produced for most of the primers. In total, the primer combinations yielded 2372 amplification products of which 563 (24%) were polymorphic. The produced and scored polymorphic bands were in general well spaced with easily scoreable bands. The amplification of each entry was carried out in duplicate and no major band discrepancies between reps were noted. Therefore, the scored and highly visible banding pattern under the described amplification conditions of this experiment is considered as being highly repeatable and reliable.

The presence or absence of DNA amplification fingerprint fragments in the pumpkin genotypes considered gave rise to a matrix used to calculate the coefficient of genetic similarity index (GSI) among the four pumpkin lines. The index values indicate the degree of genetic relationship of one entry with another. The index values in Table 10 ranged from 0.324 for hybrids Merlin and HSR4709 to 0.592 for hybrids Spartan and Merlin. As noted the degree of relatedness between the hybrids is described as the genetic similarity index value. For example, the Nei and Li (N&L) genetic similarity index for hybrids Merlin and HSR4709 is 0.324. If this is a truly random sample of DNA markers, which it is, then 95% (i.e. 95% confidence) of random 94 genetic DNA polymorphisms will be between 0.270 and 0.38 confidence limits. In Table 13, LCL means the lower confidence limit at 95% probability level and UCL means the upper confidence limit at 95% probability level.

The results in Table 12 and Table 13 demonstrated that the genetic relationship (GS=0.503) of the new pumpkin HSR4709, which contains the mutant allele Pm2 of the present invention, and PMS pumpkin Frosty were much closer than either of the commercial variety Merlin (GS=0.324) or Spartan (GS=0.362). This shows that the powdery mildew resistance displayed by HSR4709 containing the mutant allele Pm2 of the present invention was derived from a different genetic source from that of either Merlin or Spartan.

TABLE 12

| | Merlin | HSR4709 | Frosty | Spartan |
|---|---|---|---|---|
| Merlin | 1.000 | 0.324 | 0.370 | 0.592 |
| HSR4709 | 0.324 | 1.000 | 0.503 | 0.362 |
| Frosty | 0.370 | 0.503 | 1.000 | 0.467 |
| Spartan | 0.592 | 0.362 | 0.467 | 1.000 |

TABLE 13

| | | LCL | UCL |
|---|---|---|---|
| Merlin | HSR4709 | 0.27 | 0.38 |
| Merlin | Frosty | 0.31 | 0.43 |
| Merlin | Spartan | 0.53 | 0.65 |
| HSR4709 | Frosty | 0.45 | 0.56 |
| HSR4709 | Spartan | 0.31 | 0.42 |
| Frosty | Spartan | 0.41 | 0.53 |

Table 13 shows the mean separation or paired comparison for colony counts for the pumpkin varieties containing the mutant allele Pm2 of the present invention with commercial pumpkin varieties Merlin, Magic Lantern and Spartan. “z” means with the same letter are not significantly different at 0.05 level. Magic Lantern, Spartan and Merlin are commercial pumpkin varieties used as comparisons, which do not carry the mutant Pm2 allele. “y” means the treatments consist of leaves, stems and petioles.

### Example 7

### Determining the Allelic and Genetic Differences of the Mutant Allele Pm2 in The Present Invention and an Existing Powdery Mildew Resistant Allele Pm in Commercial Varieties

In order to decipher and differentiate the allelic and genetic sequence differences between the PMR mutant allele Pm2 in the present invention and an existing powdery mildew resistant allele Pm present in commercial pumpkin varieties, published sequences for powdery mildew resistance genes were analyzed and a total of 14 PCR primers linked to putative PMR genes from C. pepo and other related cucurbit species were designed and screened against pumpkin DNA. The results show that the mutant allele Pm2 of the present invention is distinct from the existing PMR allele Pm, and that mutant allele Pm2 may be contained within the PMR5 gene of C. pepo. The pumpkin lines tested included: HSPMR7B1, which contains mutant allele Pm2 of the present invention; M Line 411, which is derived from commercial variety Merlin and contains the existing PMR allele Pm; and Hdy 3111, which is a powdery mildew susceptible line. The following primers were used:

Primers from PMR5 from: Blanca et al., 2011, Transcriptome characterization and high throughput SSRs and SNPs discovery in Cucurbita pepo (Cucurbitaceae), BMC Genomics, 12:104:

(BLAST AT ICUGI for PU0055704, also EST
F50TW9202IN70A)
AgBPMR5F1: 5′ GCTTCATTCCCAGTTGGCTTCTTCAG 3′
AgBPMR5R1: 5′ CCATTCATTGATATCTCCTCTAGCTTCAGG 3′

Primers from: Yuste-Lisbona et al. 2011, Codominant PCR based markers and candidate genes for powdery mildew resistance in melon, TAG 122(4):747-758:

AgBPM5F2: 5′ CCAACTAGATTGAATAACATTCCTCAC 3′
AgBPM5R2: 5′ GATACAATTTCCAACAGCATTCTGAGG 3′
AgBPM5F3: 5′ GAGGAAATTAAAGGAAGACTTGTCACTCG 3′
AgBPM5R3: 5′ TCCTGATTTCTCTTGAGTGAGGAATG 3′
AgBPM2F1: 5′ GAATTCACTTCCGTCCCGCG 3′
AgBPM2R2: 5′ TTAAATACCTGTTTGTATTGGATTTCAATG 3′
AgBPM6F1: 5′ GAATTCAAACCACAATCTTCGTAATC 3′
AgBPM6R1: 5′ CGAGAATCAGAGAGGGTCGT 3′

Primers from Cucumis melo supsp. melo sequence HM854749:

Primers from MRGH5, a putative powdery mildew resistance gene:

Amplifies Intron 1:

AgBMRGH5F1: 5′ GAGAAGGATTGGCCAAACATGAGG 3′
AgBMRGH5R1: 5′ GTTAACACATTCAGACTTGTACATTTCTTC 3′

Amplifies Intron 2:

AgBMRGH5F2:
5′ GATATTCTTCAATTAAGTTTTGATGGGC 3′
AgBMRGH5R2:
5′ GATCTTTTGTAATAAAGCATGAGGGTAAAGATG 3′

Amplifies Intron 3:

AgBMRGH5F3:
5′ CATCTTTACCCTCATGCTTTATTACAAAAGATC 3′
AgBMRGH5R3:
5′ GCTTAGTCAGAGATCCAATAGAATCATGAATC 3′

Primers from MRGH63, a putative powdery mildew resistance gene (gene is minus sense):

Intron 2 and 3:

AgbMRGH63F1: 5′ TATTGAGAGATCCAATTGATTCATGAAC 3′
AgBMRGH63R1: 5′ AATGATGTTAGAAGCATGTGGTTG 3′

Intron 1 (Partial):

AgBMRGH63F2: 5′ CAGAAAGCAACAACCTTCAAAGTCATCAGC 3′
AgBMRGH63R2: 5′ CAAATCTTTGCTTTTTATAAAGAAATCCACC 3′

Primers from MU33816, a putative powdery mildew resistance like protein:

AgBMU33F1: 5′ GTATAAAGCTTTAGCCAAAGTTGTCTTACC 3′
AgBMU33R1: 5′ GCACCAATTTCACCAGTCATCTTGATATGG 3′

Primers from C. pepo mRNA sequence JF332040, coding for a putative powdery mildew resistance protein:

AgBJF33F1: 5′ ACTCACCTCAAGACTATGTTGATGCTCACA 3′
AgBJF33F2: 5′ CAAGACTATGTTGATGCTCACAATATTGC 3′
AgBJF33F1: 5′ ACTCACCTCAAGACTATGTTGATGCTCACA 3′
AgBJF33R1: 5′ TCATCCACCCACATTTGAACTGCTTC 3′

Primers from C. moschata mRNA sequence EU056338, coding for a chitinase induced in powdery mildew infection (SNPs with C. pepo at ICUGI site):

AgBEU05F1: 5′ CTCGCTGCTTTCTTCGGCCAAAC 3′
AgBEU05R1: 5′ GCTTGTTTCCTTGTGGTGTCATCCAG 3′

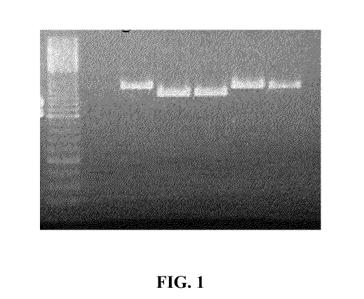

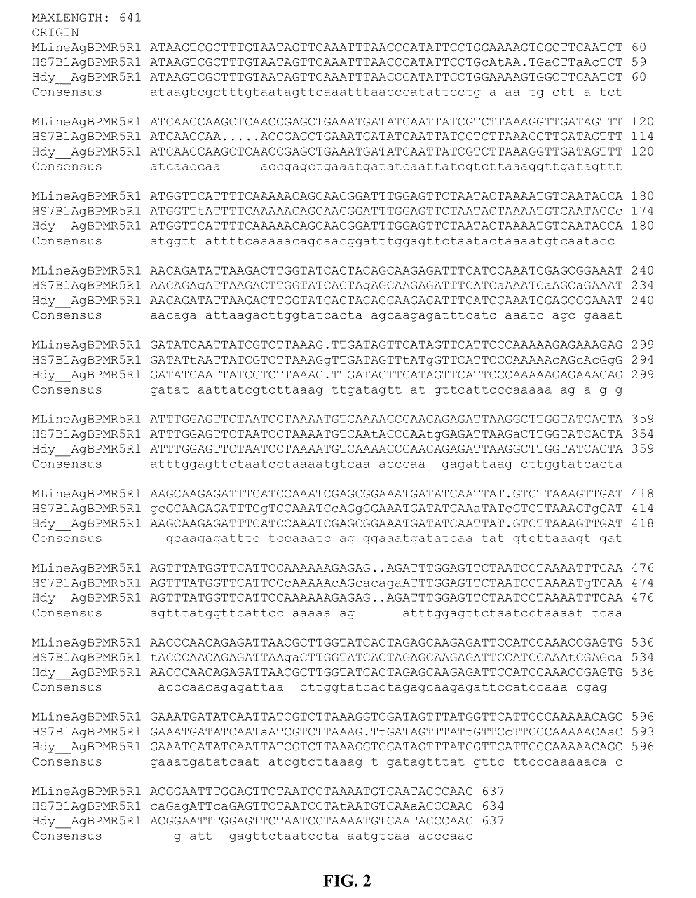

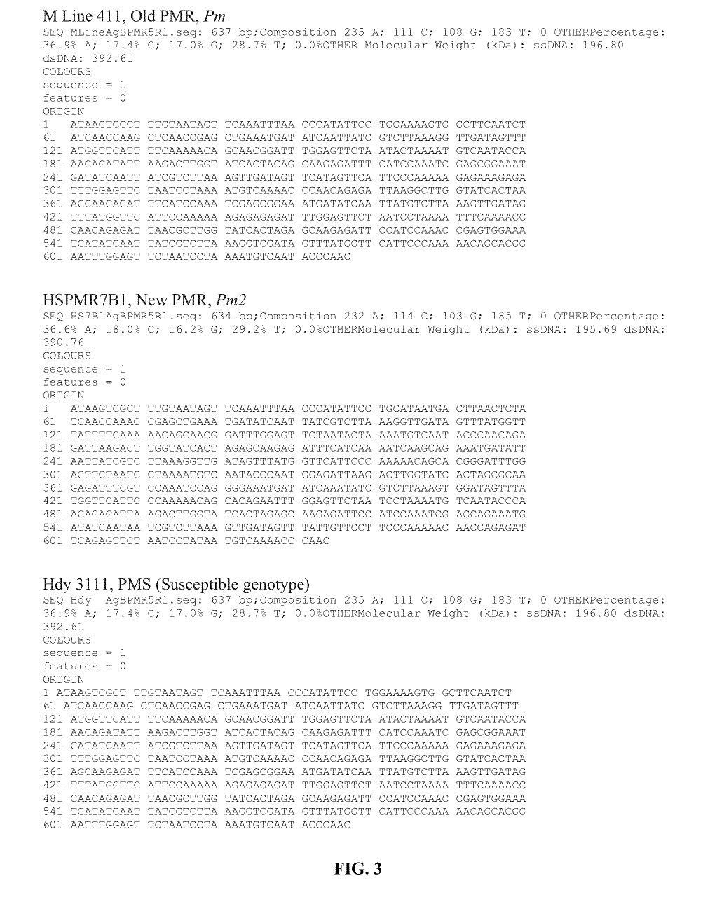

A few of the primers did not generate any amplicons, and most of the primers that did generate amplicons had the same size amplicon for each genotype. However, one putative PMR unigene primer set AgBPMP5F1/R1, derived from the PMR5 sequence from C. pepo (BLAST at ICUGI for PU055704, and also EST F50TW9202IN704), produced amplicons that displayed the same size for the resistant M line 411 and the susceptible control Hdy 3111, but produced a different amplicon size for line HSPMR7B1, which contains the mutant allele of the present invention Pm2 (see FIG. 1). This allowed allele-specific discrimination of PCR products for the different genotypes. To distinguish the genotypes at the nucleotide level, the AgBPMP5F1/R1 amplicons were subjected to DNA sequencing. Sequence alignment clearly indicated the presence of indels and multiple SNPs between the HSPMR7B1 and the M line 411 and Hdy 3111 genotypes (see FIG. 2). Sequences from the reverse primer of AgBPMP5F1/R1 are shown in FIG. 3 (see also SEQ ID NO:1-3). These results indicate that the mutant allele Pm2 of the present invention is distinct from the existing PMR allele Pm, and that mutant allele Pm2 may be contained within the PMR5 gene of C. pepo.

Thousands of Curcubita unigenes have been identified with orthologs in Arabidopsis, including an ortholog to Arabidopsis PMR5 (powdery mildew resistant 5; AT5G58600) (Blanca et al., 2011, Transcriptome characterization and high throughput SSRs and SNPs discovery in Cucurbita pepo (Cucurbitaceae), BMC Genomics, 12:104). PMR5 has been identified as being involved in resistance to powdery mildew infection. A mutation in the pmr5 gene rendered Arabidopsis resistant to the powdery mildew species Erysiphe cichoracearum and Erysiphe orontii. Studies by Vogel et al. demonstrated that pmr5-mediated resistance is independent of the activation of known defense pathways, and therefore PMR5 is either required for fungal growth or the pmr5 mutation activates a novel defense pathway. (Vogel et al., 2004, Mutations in PMR5 result in powdery mildew resistance and altered cell wall composition, The Plant Journal, 40:968-978).

The sequence alignment of the AgBPMP5F1/R1 amplicons (see FIG. 2) show the presence of indels and multiple SNPs between HSPMR7B1 and the M line 411 and Hdy 3111 genotypes in the PMR5 gene. These differences, together with the documented role of PMR5 in powdery mildew, show that mutant allele Pm2 of the present invention may be contained within PMR5.

### FURTHER EMBODIMENTS OF THE INVENTION

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes.” Over the last fifteen to twenty years several methods for producing transgenic plants have been developed and the present invention, in particular embodiments, also relates to transformed versions of the claimed variety or line.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid and can be used alone or in combination with other plasmids to provide transformed C. pepo plants using transformation methods as described below to incorporate transgenes into the genetic material of the C. pepo plant(s).

Expression Vectors for C. pepo Transformation: Marker Genes

Expression vectors include at least one genetic marker operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. USA, 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant (Hayford et al., Plant Physiol. 86:1216 (1988), Jones et al., Mol. Gen. Genet., 210:86 (1987), Svab et al., Plant Mol. Biol. 14:197 (1990), Hille et al., Plant Mol. Biol. 7:171 (1986)). Other selectable marker genes confer resistance to herbicides such as glyphosate, glufosinate or bromoxynil (Comai et al., Nature 317:741-744 (1985), Gordon-Kamm et al., Plant Cell 2:603-618 (1990) and Stalker et al., Science 242:419-423 (1988)).

Selectable marker genes for plant transformation not of bacterial origin include, for example, mouse dihydrofolate reductase, plant 5-enolpyruvylshikimate-3-phosphate synthase and plant acetolactate synthase (Eichholtz et al., Somatic Cell Mol. Genet. 13:67 (1987), Shah et al., Science 233:478 (1986), Charest et al., Plant Cell Rep. 8:643 (1990)).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase and chloramphenicol acetyltransferase (Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984)).

In vivo methods for visualizing GUS activity that do not require destruction of plant tissue are available (Molecular Probes publication 2908, IMAGENE GREEN, pp. 1-4 (1993) and Naleway et al., J. Cell Biol. 115:151a (1991)). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells (Chalfie et al., Science 263:802 (1994)). GFP and mutants of GFP may be used as screenable markers.

Expression Vectors for C. pepo Transformation: Promoters

Genes included in expression vectors must be driven by a nucleotide sequence comprising a regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific.” A “cell-type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions.

A. Inducible Promoters:

An inducible promoter is operably linked to a gene for expression in C. pepo. Optionally, the inducible promoter is operably linked to a nucleotide sequence encoding a signal sequence which is operably linked to a gene for expression in C. pepo. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Mett et al., Proc. Natl. Acad. Sci. USA 90:4567-4571 (1993)); In2 gene from maize which responds to benzenesulfonamide herbicide safeners (Hershey et al., Mol. Gen. Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. USA 88:0421 (1991)).

B. Constitutive Promoters:

A constitutive promoter is operably linked to a gene for expression in C. pepo or the constitutive promoter is operably linked to a nucleotide sequence encoding a signal sequence which is operably linked to a gene for expression in C. pepo.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)). The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT publication number WO 96/30530.

C. Tissue-Specific or Tissue-Preferred Promoters:

A tissue-specific promoter is operably linked to a gene for expression in C. pepo. Optionally, the tissue-specific promoter is operably linked to a nucleotide sequence encoding a signal sequence which is operably linked to a gene for expression in C. pepo. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplast. Many signal sequences are known in the art. See, for example, Becker et al., Plant Mol. Biol. 20:49 (1992); Close, P. S., Master's Thesis, Iowa State University (1993); Knox, C., et al., Plant Mol. Biol. 9:3-17 (1987); Lerner et al., Plant Physiol. 91:124-129 (1989); Frontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant J. 2:129 (1991); Kalderon, et al., Cell 39:499-509 (1984); Steifel, et al., Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is a C. pepo plant. In another preferred embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology, CRC Press, Boca Raton 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

A. Genes that Confer Resistance to Pests or Disease and that Encode:

1. Plant disease resistance genes. Plant defenses are often activated by specific interaction between the product of a disease resistance gene (R) in the plant and the product of a corresponding avirulence (Avr) gene in the pathogen. A plant variety can be transformed with one or more cloned resistance genes to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al. Cell 78:1089 (1994) (Arabidopsis RSP2 gene for resistance to Pseudomonas syringae).

2. A gene conferring resistance to a pest, such as soybean cyst nematode. See e.g., PCT Publication Number WO 96/30517; PCT Publication Number WO 93/19181.

3. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Numbers 40098, 67136, 31995, and 31998.

4. A lectin. See, for example, Van Damme et al., Plant Molec. Biol. 24:25 (1994), who disclose the nucleotide sequences of several Clivia miniata mannose-binding lectin genes.

5. A vitamin-binding protein such as avidin. See PCT Application Number U.S. 93/06487 which teaches the use of avidin and avidin homologues as larvicides against insect pests.

6. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotech. Biochem. 57:1243 (1993) (nucleotide sequence of Streptomyces nitrosporeus α-amylase inhibitor) and U.S. Pat. No. 5,494,813 (Hepher and Atkinson, issued Feb. 27, 1996).

7. An insect-specific hormone or pheromone such as an ecdysteroid or juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

8. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., which discloses genes encoding insect-specific, paralytic neurotoxins.

9. An insect-specific venom produced in nature by a snake, a wasp, etc. For example, see Pang et al., Gene 116:165 (1992), for disclosure of heterologous expression in plants of a gene coding for a scorpion insectotoxic peptide.

10. An enzyme responsible for a hyperaccumulation of a monoterpene, a sesquiterpene, a steroid, hydroxamic acid, a phenylpropanoid derivative or another non-protein molecule with insecticidal activity.

11. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT Publication WO 93/02197 (Scott et al.), which discloses the nucleotide sequence of a callase gene. DNA molecules which contain chitinase-encoding sequences can be obtained, for example, from the ATCC under Accession Numbers 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene.

12. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

13. A hydrophobic moment peptide. See PCT Publication WO 95/16776, which discloses peptide derivatives of tachyplesin which inhibit fungal plant pathogens, and PCT Publication WO 95/18855 which teaches synthetic antimicrobial peptides that confer disease resistance.

14. A membrane permease, a channel former or a channel blocker. For example, see the disclosure of Jaynes et al., Plant Sci 89:43 (1993), of heterologous expression of a cecropin-β lytic peptide analog to render transgenic tobacco plants resistant to Pseudomonas solanacearum.

15. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus and tobacco mosaic virus.

16. An insect-specific antibody or an immunotoxin derived therefrom. Thus, an antibody targeted to a critical metabolic function in the insect gut would inactivate an affected enzyme, killing the insect. See Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

17. A virus-specific antibody. See, for example, Tavladoraki et al., Nature 366:469 (1993), who show that transgenic plants expressing recombinant antibody genes are protected from virus attack.

18. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

19. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

20. Genes involved in the Systemic Acquired Resistance (SAR) Response and/or the pathogenesis-related genes. Briggs, S., Current Biology, 5(2) (1995).

21. Antifungal genes. See Cornelissen and Melchers, Plant Physiol., 101:709-712 (1993); Parijs et al., Planta 183:258-264 (1991) and Bushnell et al., Can. J. of Plant Path. 20(2):137-149 (1998).

22. Genes that confer resistance to Phytophthora root rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker et al., Phytophthora Root Rot Resistance Gene Mapping in Soybean, Plant Genome IV Conference, San Diego, Calif. (1995).

B. Genes that Confer Resistance to an Herbicide, for Example:

1. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

2. Glyphosate (resistance conferred by mutant 5-enolpyruvlshikimate-3-phosphate synthase (EPSPS) and aroA genes, respectively) and other phosphono compounds such as glufosinate (phosphinothricin acetyl transferase (PAT) and Streptomyces hygroscopicus PAT bar genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. A DNA molecule encoding a mutant aroA gene can be obtained under ATCC Accession Number 39256, and the nucleotide sequence of the mutant gene is disclosed in U.S. Pat. No. 4,769,061 to Comai. European Patent Application Number 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European Application Number 0 242 246 to Leemans et al. DeGreef et al., Bio/Technology 7:61 (1989) describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2, and Acc2-S3 genes described by Marshall et al., Theor. Appl. Genet. 83:435 (1992).

3. An herbicide that inhibits photosynthesis, such as a triazine (psbA and gs+ genes) and a benzonitrile (nitrilase gene). Przibila et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Numbers 53435, 67441, and 67442. Cloning and expression of DNA coding for a glutathione S-transferase is described by Hayes et al., Biochem. J. 285:173 (1992).

4. Acetohydroxy acid synthase, which has been found to make plants that express this enzyme resistant to multiple types of herbicides, has been introduced into a variety of plants. See Hattori et al., Mol. Gen. Genet. 246:419, 1995. Other genes that confer tolerance to herbicides include a gene encoding a chimeric protein of rat cytochrome P4507A1 and yeast NADPH-cytochrome P450 oxidoreductase (Shiota et al., Plant Physiol., 106:17, 1994), genes for glutathione reductase and superoxide dismutase (Aono et al., Plant Cell Physiol. 36:1687, 1995), and genes for various phosphotransferases (Datta et al., Plant Mol. Biol. 20:619, 1992).

5. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; 5,767,373; and PCT Publication WO 01/12825.

C. Genes that Confer or Contribute to a Value-Added Trait, Such as:

1. Modified fatty acid metabolism, for example, by transforming a plant with an antisense gene of stearyl-ACP desaturase to increase stearic acid content of the plant. See Knultzon et al., Proc. Natl. Acad. Sci. USA 89:2625 (1992).

2. Decreased phytate content—1) Introduction of a phytase-encoding gene enhances breakdown of phytate, adding more free phosphate to the transformed plant. For example, see Van Hartingsveldt et al., Gene 127:87 (1993), for a disclosure of the nucleotide sequence of an Aspergillus niger phytase gene. 2) A gene could be introduced that reduced phytate content. This could be accomplished by cloning and then reintroducing DNA associated with the single allele which is responsible for maize mutants characterized by low levels of phytic acid. See Raboy et al., Maydica 35:383 (1990).

3. Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus lichenifonnis α-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley α-amylase gene), and Fisher et al., Plant Physiol. 102:1045 (1993) (maize endosperm starch branching enzyme II).

4. Elevated oleic acid via FAD-2 gene modification and/or decreased linolenic acid via FAD-3 gene modification. See U.S. Pat. Nos. 6,063,947; 6,323,392; and PCT Publication WO 93/11245.

D. Genes that Control Male Sterility:

1. Introduction of a deacetylase gene under the control of a tapetum-specific promoter and with the application of the chemical N-Ac-PPT. See PCT Publication WO 01/29237.

2. Introduction of various stamen-specific promoters. See PCT Publications WO 92/13956 and WO 92/13957.

3. Introduction of the barnase and the barstar genes. See Paul et al., Plant Mol. Biol. 19:611-622, 1992).

Methods for C. pepo Transformation

Numerous methods for plant transformation have been developed including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

A. Agrobacterium-Mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra and Moloney et al., Plant Cell Reports 8:238 (1989). See also, U.S. Pat. No. 5,563,055 (Townsend and Thomas), issued Oct. 8, 1996.

B. Direct Gene Transfer

Several methods of plant transformation, collectively referred to as direct gene transfer, have been developed as an alternative to Agrobacterium-mediated transformation. A generally applicable method of plant transformation is microprojectile-mediated transformation where DNA is carried on the surface of microprojectiles measuring 1 μm to 4 μm. The expression vector is introduced into plant tissues with a biolistic device that accelerates the microprojectiles to speeds of 300 m/s to 600 m/s which is sufficient to penetrate plant cell walls and membranes. Sanford et al., Part. Sci. Technol. 5:27 (1987); Sanford, J. C., Trends Biotech. 6:299 (1988); Klein et al., Bio/Tech. 6:559-563 (1988); Sanford, J. C. Physiol Plant 7:206 (1990); Klein et al., Biotechnology 10:268 (1992). See also U.S. Pat. No. 5,015,580 (Christou, et al.), issued May 14, 1991 and U.S. Pat. No. 5,322,783 (Tomes, et al.), issued Jun. 21, 1994.

Another method for physical delivery of DNA to plants is sonication of target cells. Zhang et al., Bio/Technology 9:996 (1991). Alternatively, liposome and spheroplast fusion have been used to introduce expression vectors into plants. Deshayes et al., EMBO J., 4:2731 (1985); Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described (Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994)).

Following transformation of C. pepo target tissues, expression of the above-described selectable marker genes allows for preferential selection of transformed cells, tissues and/or plants, using regeneration and selection methods well known in the art.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular C. pepo line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple X by Y cross or the process of backcrossing depending on the context.

Gene Conversions

When the term “C. pepo plant” is used in the context of the present invention, this also includes any single gene conversions of that variety. The term single gene converted or gene converted plant as used herein refers to those C. pepo plants which are developed by a plant breeding technique called backcrossing wherein essentially all of the desired morphological and physiological characteristics of a variety are recovered in addition to the single gene transferred into the variety via the backcrossing technique. Backcrossing methods can be used with the present invention to improve or introduce a characteristic into the variety. The term “backcrossing” as used herein refers to the repeated crossing of a hybrid progeny back to the recurrent parent, i.e., backcrossing 1, 2, 3, 4, 5, 6, 7, 8 or more times to the recurrent parent. The parental C. pepo plant that contributes the gene for the desired characteristic is termed the nonrecurrent or donor parent. This terminology refers to the fact that the nonrecurrent parent is used one time in the backcross protocol and therefore does not recur. The parental C. pepo plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original variety of interest (recurrent parent) is crossed to a second variety (nonrecurrent parent) that carries the single gene or genes of interest to be transferred. The resulting progeny from this cross are then crossed again to the recurrent parent and the process is repeated until a C. pepo plant is obtained wherein essentially all of the desired morphological and physiological characteristics of the recurrent parent are recovered in the converted plant, in addition to the single transferred gene from the nonrecurrent parent.

The selection of a suitable recurrent parent is an important step for a successful backcrossing procedure. The goal of a backcross protocol is to alter or substitute a single trait or characteristic in the original variety. To accomplish this, a single gene of the recurrent variety is modified or substituted with the desired gene from the nonrecurrent parent, while retaining essentially all of the rest of the desired genetic, and therefore the desired physiological and morphological, constitution of the original variety. The choice of the particular nonrecurrent parent will depend on the purpose of the backcross; one of the major purposes is to add some agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single or multiple gene traits have been identified that are not regularly selected for in the development of a new variety but that can be improved by backcrossing techniques. Single gene traits may or may not be transgenic; examples of these traits include but are not limited to, male sterility, waxy starch, herbicide resistance, resistance for bacterial, fungal, or viral disease, insect resistance, male fertility, enhanced nutritional quality, industrial usage, yield stability and yield enhancement. These genes are generally inherited through the nucleus. Several of these single gene traits are described in U.S. Pat. Nos. 5,959,185; 5,973,234 and 5,977,445; the disclosures of which are specifically hereby incorporated by reference.

Tissue Culture

Further reproduction of a variety can occur by tissue culture and regeneration. Tissue culture of various tissues of C. pepo and regeneration of plants therefrom is well known and widely published. For example, reference may be had to Jelaska, S. et al., Physiol. Plant. 64(2):237-242 (1985) and Krsnik-Rasol, M., Int. J. Dev. Biol. 35(3):259-263 (1991). Thus, another aspect of this invention is to provide cells which upon growth and differentiation produce C. pepo plants having the mutant allele Pm2.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, pistils and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

Additional Breeding Methods

This invention also is directed to methods for producing a C. pepo plant by crossing a first parent C. pepo plant with a second parent C. pepo plant wherein the first or second parent C. pepo plant is a C. pepo plant comprising the mutant allele Pm2. Further, both first and second parent C. pepo plants can comprise the mutant allele Pm2. Thus, any such methods using a C. pepo plant comprising the mutant allele Pm2 are part of this invention: selfing, backcrosses, hybrid production, crosses to populations, and the like.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max L. Men.) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD, three classical markers and four isozyme loci. See also, Shoemaker, R. C., RFLP Map of Soybean, p 299-309, in Phillips, R. L. and Vasil, I. K., Eds. DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which includes markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into pumpkin and squash varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogues like 5-bromo-uracil), antibiotics, alkylating agents (such as sulfur mustards, nitrogen mustards, epoxides, ethylene amines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

Descriptions of other breeding methods that are commonly used for different traits and crops can be found in one of several reference books (e.g., Allard, 1960; Simmonds, 1979; Sneep et al., 1979; Fehr, 1987).

### DEPOSIT INFORMATION

A deposit of the Hollar Seeds proprietary pumpkin variety designated HSPMR7B1 containing the Pm2 mutant allele of the present invention disclosed above and recited in the appended claims has been made with the American Type Culture Collection (ATCC), 10801 University Boulevard, Manassas, Va. 20110. The date of deposit was Aug. 6, 2010. The deposit of 2,500 seeds was taken from the same deposit maintained by Hollar Seeds since prior to the filing date of this application. All restrictions upon the deposit will be irrevocably removed upon granting of a patent, and the deposit is intended to meet all of the requirements of 37 C.F.R. §§1.801-1.809. The ATCC Accession Number is PTA-11250. The deposit will be maintained in the depository for a period of 30 years, or 5 years after the last request, or for the effective life of the patent, whichever is longer, and will be replaced as necessary during that period.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A Cucurbita pepo plant containing mutant allele Pm2, wherein said plant has increased resistance and consistent resistance to powdery mildew.

2. A Cucurbita pepo seed containing mutant allele designated Pm2, wherein said mutant allele Pm2 confers increased resistance and consistent resistance to powdery mildew, and wherein a representative sample of said seed containing said mutant allele Pm2 has been deposited under ATCC Accession No. PTA-11250.

3. A Cucurbita pepo plant, or a part thereof, produced by growing the C. pepo seed of claim 2.

4. A tissue culture of cells produced from the plant of claim 3, wherein said cells of the tissue culture are produced from a plant part selected from the group consisting of leaf, pollen, embryo, cotyledon, hypocotyl, meristematic cell, root, root tip, pistil, anther, flower, stem, fruit and petiole.

5. A protoplast produced from the plant of claim 3.

6. A protoplast produced from the tissue culture of claim 4.

7. A C. pepo plant regenerated from the tissue culture of claim 4, wherein said plant has increased resistance and consistent resistance to powdery mildew.

8. A method for producing an F₁ hybrid C. pepo seed, wherein the method comprises crossing the plant of claim 3 with a different C. pepo plant and harvesting the resultant F₁ hybrid C. pepo seed.

9. A hybrid C. pepo seed produced by the method of claim 8.

10. A hybrid C. pepo plant, or a part thereof, produced by growing said hybrid seed of claim 9, wherein said plant contains mutant allele Pm2, and wherein said mutant allele confers increased resistance and consistent resistance to powdery mildew.

11. A method for transferring mutant allele Pm2 to a different genetic background, wherein the method comprises:
a) obtaining the F₁ plant of claim 10;
b) backcrossing said F₁ plant to a recipient parent plant not having mutant allele Pm2 to produce backcross progeny plants;
c) selecting for backcross progeny plants that contain mutant allele Pm2;
d) backcrossing said selected backcross progeny plants to said recipient parent;
e) repeating steps (c) and (d) two or more time in succession to produce selected third or higher backcross progeny plants that contain mutant allele Pm2; and
f) harvesting the resulting seed.

12. A plant produced from the seed of claim 11, wherein said plant contains mutant allele Pm2 and has increased resistance and consistent resistance to powdery mildew.


SEQUENCE LISTING (3 sequences)

SEQ ID NO: 1 - DNA, 637 bp, Cucurbita pepo
ataagtcgctttgtaatagttcaaatttaacccatattcctggaaaagtggcttcaatctatcaaccaagctcaaccgagctgaaatgatatcaattatcgtcttaaaggttgatagtttatggttcattttcaaaaacagcaacggatttggagttctaatactaaaatgtcaataccaaacagatattaagacttggtatcactacagcaagagatttcatccaaatcgagcggaaatgatatcaattatcgtcttaaagttgatagttcatagttcattcccaaaaagagaaagagatttggagttctaatcctaaaatgtcaaaacccaacagagattaaggcttggtatcactaaagcaagagatttcatccaaatcgagcggaaatgatatcaattatgtcttaaagttgatagtttatggttcattccaaaaaagagagagatttggagttctaatcctaaaatttcaaaacccaacagagattaacgcttggtatcactagagcaagagattccatccaaaccgagtggaaatgatatcaattatcgtcttaaaggtcgatagtttatggttcattcccaaaaacagcacggaatttggagttctaatcctaaaatgtcaatacccaac

SEQ ID NO: 2 - DNA, 634 bp, Cucurbita pepo
ataagtcgctttgtaatagttcaaatttaacccatattcctgcataatgacttaactctatcaaccaaaccgagctgaaatgatatcaattatcgtcttaaaggttgatagtttatggtttattttcaaaaacagcaacggatttggagttctaatactaaaatgtcaatacccaacagagattaagacttggtatcactagagcaagagatttcatcaaaatcaagcagaaatgatattaattatcgtcttaaaggttgatagtttatggttcattcccaaaaacagcacgggatttggagttctaatcctaaaatgtcaatacccaatggagattaagacttggtatcactagcgcaagagatttcgtccaaatccaggggaaatgatatcaaatatcgtcttaaagtggatagtttatggttcattcccaaaaacagcacagaatttggagttctaatcctaaaatgtcaatacccaacagagattaagacttggtatcactagagcaagagattccatccaaatcgagcagaaatgatatcaataatcgtcttaaagttgatagtttattgttccttcccaaaaacaaccagagattcagagttctaatcctataatgtcaaaacccaac

SEQ ID NO: 3 - DNA, 637 bp, Cucurbita pepo; identical to SEQ ID NO: 1